(19) United States
(12) Patent Application Publication
(10) Pub. No.: US 2022/0054330 A1
(43) Pub. Date: Feb. 24, 2022

(54) MOVEABLE MEDICAL DEVICES AND MONITORING METHODS THEREOF

(71) Applicant: SHANGHAI UNITED IMAGING HEALTHCARE CO., LTD., Shanghai (CN)

(72) Inventors: Xiaozhen LIN, Shanghai (CN); Daheng CHEN, Shanghai (CN); Wei HU, Shanghai (CN); Zhouyuan FAN, Shanghai (CN); Yifeng JIANG, Shanghai (CN); Yangyang LIN, Shanghai (CN); Pei ZHOU, Shanghai (CN); Tingting WU, Shanghai (CN)

(73) Assignee: SHANGHAI UNITED IMAGING HEALTHCARE CO., LTD., Shanghai (CN)

(21) Appl. No.: 17/445,726

(22) Filed: Aug. 23, 2021

(30) Foreign Application Priority Data
Aug. 21, 2020 (CN) 202010850685.4
Nov. 11, 2020 (CN) 202011257120.1
Nov. 11, 2020 (CN) 202022599961.2
Dec. 16, 2020 (CN) 202011489934.8

(51) Int. Cl. A61G 3/00; A61B 6/00; A61B 6/04; A61G 10/00; A61L 2/10; A61L 2/22; G07C 9/37

(52) U.S. Cl. CPC A61G 3/001; A61L 2202/14; A61B 6/54; A61B 6/0407; A61B 6/4423; A61B 6/56; A61G 10/00; A61L 2/10; A61L 2/22; G07C 9/37; A61G 2210/50; A61L 2202/25; A61L 2202/11; A61L 2202/16; A61B 6/4405

(57) ABSTRACT

A movable medical device is provided. The movable medical device may include a medical cabin configured to accommodate medical resources for providing multiple types of medical services and a movable platform configured to move the medical cabin to a desired location. The medical cabin may include a radiation room configured to accommodate a radiation device for providing a medical imaging service; a sample processing room configured to accommodate medical resources for providing at least one of a sampling service or a sample detection service; and a control room configured to accommodate a control platform used to control the radiation device for providing the medical imaging service.

### CROSS-REFERENCE TO RELATED APPLICATIONS

This application claims priority to Chinese Patent Application No. 202011257120.1, filed on Nov. 11, 2020, Chinese Patent Application No. 202022599961.2, filed on Nov. 11, 2020, Chinese Patent Application No. 202010850685.4, filed on Aug. 21, 2020, and Chinese Patent Application No. 202011489934.8, filed on Dec. 16, 2020, the contents of each of which are hereby incorporated by reference.

### TECHNICAL FIELD

This disclosure generally relates to medical technology, and more particularly, relates to movable medical devices and methods and systems for monitoring and/or powering a movable medical device.

### BACKGROUND

With the development of medical equipment technology, some auxiliary diagnostic equipment, such as computed tomography (CT), has become an important auxiliary way of disease diagnosis. This auxiliary diagnostic equipment is usually set up in the hospital and requires patients to go to the hospital for examination. However, in some special occasions, this auxiliary diagnostic equipment needs to be moved to a specific location, which is not convenient to move and causes other problems, such as power supply, the influence of the environment on the auxiliary diagnostic equipment, etc. Therefore, it is desirable to provide movable medical devices, and methods and systems for monitoring and/or powering a movable medical device.

### SUMMARY

In an aspect of the present disclosure, a movable medical device is provided. The movable medical device may include a medical cabin configured to accommodate medical resources for providing multiple types of medical services and a movable platform configured to move the medical cabin to a desired location. The medical cabin may include a radiation room configured to accommodate a radiation device for providing a medical imaging service; a sample processing room configured to accommodate medical resources for providing at least one of a sampling service or a sample detection service; and a control room configured to accommodate a control platform used to control the radiation device for providing the medical imaging service.

In another aspect of the present disclosure, a movable medical device is provided. The movable medical device may include a medical cabin configured to accommodate medical resources for providing one or more types of medical services and a movable platform configured to move the medical cabin to a desired location. The medical cabin may include a power supply system configured to provide power for a load in the movable medical device. The power supply system may include a first electric source system and a second electric source system configured to provide power to the load; and a control component configured to determine an operation state of at least one of the first electric source system or the second electric source system, and control an operation of the at least one of the first electric source system or the second electric source system based on operation state.

In still another aspect of the present disclosure, a movable medical device is provided. The movable medical device may include a medical cabin configured to accommodate medical resources for providing one or more types of medical services; a movable platform configured to move the medical cabin to a desired location; and a monitoring system including one or more monitoring devices and a control device. The one or more monitoring devices may be configured to acquire a monitoring result related to at least one of an operation state of at least one of components of the movable medical device or an environment parameter where the medical cabin is located. The control device may be configured to provide a feedback based on the monitoring result.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments. These exemplary embodiments are described in detail with reference to the drawings. The drawings are not to scale. These embodiments are non-limiting exemplary embodiments, in which like reference numerals represent similar structures throughout the several views of the drawings, and wherein:

FIG. 1 is a schematic diagram illustrating an exemplary movable medical system according to some embodiments of the present disclosure;

FIG. 2 is a schematic diagram illustrating an exemplary movable medical device according to some embodiments of the present disclosure;

FIG. 3 is a schematic diagram illustrating an exemplary movable medical device according to some embodiments of the present disclosure;

FIG. 4 is a schematic diagram illustrating an exemplary electrical connection according to some embodiments of the present disclosure;

FIG. 5 is a block diagram illustrating an exemplary monitoring system of a movable medical device according to some embodiments of the present disclosure;

FIG. 6 is a schematic diagram illustrating an exemplary structure of a control device according to some embodiments of the present disclosure;

FIG. 7 is a flowchart illustrating an exemplary process for monitoring a movable medical device according to some embodiments of the present disclosure;

FIG. 8 is a block diagram illustrating an exemplary control device according to some embodiments of the present disclosure;

FIG. 9 is a schematic diagram illustrating an exemplary movable medical device according to some embodiments of the present disclosure;

FIG. 10 is a schematic diagram illustrating another exemplary movable medical device according to some embodiments of the present disclosure;

FIG. 11 is a schematic diagram illustrating still another exemplary movable medical device according to some embodiments of the present disclosure;

FIG. 12 is a schematic diagram illustrating a rear view of a medical cabin according to some embodiments of the present disclosure;

FIG. 13 is a schematic diagram illustrating an isolation wall according to some embodiments of the present disclosure;

FIG. 14 is a schematic diagram illustrating a sample processing room according to some embodiments of the present disclosure;

FIG. 15 is a schematic diagram illustrating an internal structure of a sample processing room according to some embodiments of the present disclosure;

FIG. 16 is a schematic diagram illustrating an external structure of a sample processing room according to some embodiments of the present disclosure;

FIG. 17 is a block diagram illustrating an exemplary processing device according to some embodiments of the present disclosure;

FIG. 18 is a schematic diagram illustrating an exemplary movable medical device according to some embodiments of the present disclosure;

FIGS. 19A-C are schematic diagram illustrating another exemplary movable medical device to some embodiments of the present disclosure;

FIG. 20 is a schematic diagram illustrating still another exemplary movable medical device according to some embodiments of the present disclosure; and

FIG. 21 is a flowchart illustrating an exemplary process for power supply according to some embodiments of the present disclosure.

### DETAILED DESCRIPTION

In the following detailed description, numerous specific details are set forth by way of examples to provide a thorough understanding of the relevant disclosure. However, it should be apparent to those skilled in the art that the present disclosure may be practiced without such details. In other instances, well-known methods, procedures, systems, components, and/or circuitry have been described at a relatively high level, without detail, to avoid unnecessarily obscuring aspects of the present disclosure. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but to be accorded the widest scope consistent with the claims.

The terminology used herein is to describe particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Also, the term “exemplary” is intended to refer to an example or illustration.

It will be understood that the terms “system,” “engine,” “unit,” “module,” and/or “block” used herein are one method to distinguish different components, elements, parts, sections, or assembly of different levels in ascending order. However, the terms may be displaced by another expression if they achieve the same purpose.

Generally, the word “module,” “unit,” or “block,” as used herein, refers to logic embodied in hardware or firmware, or to a collection of software instructions. A module, a unit, or a block described herein may be implemented as software and/or hardware and may be stored in any type of non-transitory computer-readable medium or another storage device. In some embodiments, a software module/unit/block may be compiled and linked into an executable program. It will be appreciated that software modules can be callable from other modules/units/blocks or themselves, and/or may be invoked in response to detected events or interrupts. Software modules/units/blocks configured for execution on computing devices may be provided on a computer-readable medium, such as a compact disc, a digital video disc, a flash drive, a magnetic disc, or any other tangible medium, or as a digital download (and can be originally stored in a compressed or installable format that needs installation, decompression, or decryption before execution). Such software code may be stored, partially or fully, on a storage device of the executing computing device, for execution by the computing device. Software instructions may be embedded in firmware, such as an EPROM. It will be further appreciated that hardware modules/units/blocks may be included in connected logic components, such as gates and flip-flops, and/or can be included of programmable units, such as programmable gate arrays or processors. The modules/units/blocks or computing device functionality described herein may be implemented as software modules/units/blocks but may be represented in hardware or firmware. In general, the modules/units/blocks described herein refer to logical modules/units/blocks that may be combined with other modules/units/blocks or divided into sub-modules/sub-units/sub-blocks despite their physical organization or storage. The description may be applicable to a system, an engine, or a portion thereof.

It will be understood that, although the terms “first,” “second,” “third,” etc., may be used herein to describe various elements, these elements should not be limited by these terms. These terms are only used to distinguish one element from another. For example, a first element could be termed a second element, and, similarly, a second element could be termed a first element, without departing from the scope of exemplary embodiments of the present disclosure. The term “image” in the present disclosure is used to refer to images of various forms, including a 2-dimensional image, a 3-dimensional image, a 4-dimensional image, etc.

Spatial and functional relationships between elements are described using various terms, including “connected,” “attached,” and “mounted.” Unless explicitly described as being “direct,” when a relationship between first and second elements is described in the present disclosure, that relationship includes a direct relationship where no other intervening elements are present between the first and second elements, and also an indirect relationship where one or more intervening elements are present (either spatially or functionally) between the first and second elements. In contrast, when an element is referred to as being “directly” connected, attached, or positioned to another element, there are no intervening elements present. Other words used to describe the relationship between elements should be interpreted in a like fashion (e.g., “between,” versus “directly between,” “adjacent,” versus “directly adjacent,” etc.).

Some embodiments of the present disclosure provide a movable medical device. The movable medical device may include a movable platform and a medical cabin. The movable platform may be configured to move the medical cabin to a desired location. Therefore, the movable medical device may be moved to the desired location in time, which may easily and quickly complete an equipment deployment, and solve emergency needs. The medical cabin may be configured to accommodate medical resources for providing multiple types of medical services. For example, a subject may be scanned and sampled via the movable medical device. A detection result of the subject may be determined in time, which may quickly and accurately screen out the infected. Therefore, corresponding measures (e.g., the isolation, the treatment, etc.) may be performed in time to effectively prevent a further expansion of infection disease.

In addition, the movable medical device may include a monitoring system including one or more monitoring devices, and a control device. The one or more monitoring devices may be configured to acquire a monitoring result related to at least one of an operation state of at least one of components of the movable medical device or an environment parameter where the medical cabin is located. The control device may be configured to provide feedback based on the monitoring result. Therefore, a damage to the medical resource (e.g., medical components) caused by an environmental anomaly during the movement of the movable medical device may be avoided.

Further, the movable medical device may include a power supply system. The power supply system may be configured to provide power for a load in the movable medical device. The power supply system may include a first electric source system, a second electric source system, and a control component (or a control device). The control component may be electrically connected with the first electric source system and the second electric source system, respectively. The adjustment module may be electrically connected with the load and the control component. The control module 1744 may determine whether an operation state of the first electric source system includes an anomaly based on the acquired data. In response to determining that the first electric source system does not include an anomaly, the control module may cause the first electric source system to power the load. In response to determining that the first electric source system includes an anomaly, the control module may cause the second electric source system to power the load. Accordingly, the power supply system may reduce the probability of power failure of the load and ensure the load operates stably, thereby avoiding damage to the load caused by the power failure of the load.

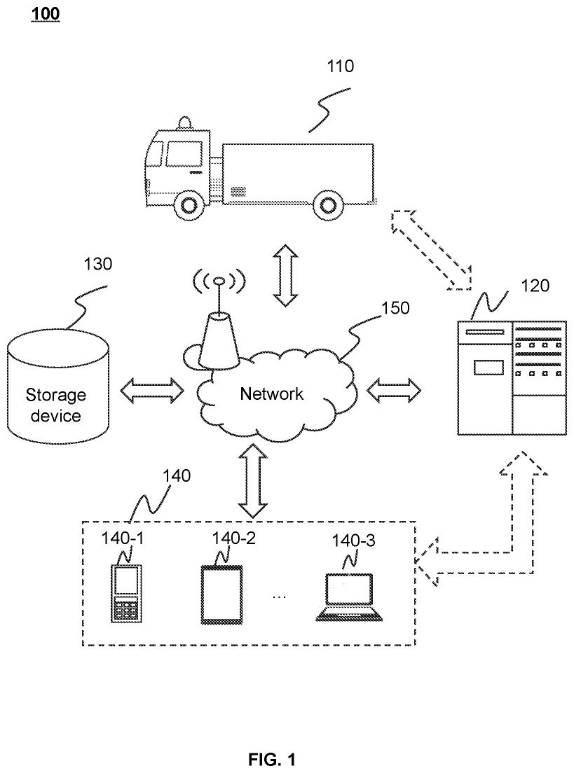

FIG. 1 is a schematic diagram illustrating an exemplary movable medical system 100 according to some embodiments of the present disclosure. As illustrated, the movable medical system 100 may include a movable medical device 110, a processing device 120, a storage device 130, a terminal 140, and a network 150. The components of the movable medical system 100 may be connected in one or more of various ways. Merely by way of example, as illustrated in FIG. 1, the movable medical device 110 may be connected to the processing device 120 directly as indicated by the bi-directional arrow in dotted lines linking the movable medical device 110 and the processing device 120, or through the network 150. As another example, the storage device 130 may be connected to the movable medical device 110 through the network 150. As still another example, the terminal 140 may be connected to the processing device 120 directly as indicated by the bi-directional arrow in dotted lines linking the terminal 140 and the processing device 120, or through the network 150.

The movable medical device 110 may include a medical cabin and a movable platform. The medical cabin may be configured to accommodate medical resources for providing one or more types of medical services. The multiple types of medical services may include a medical imaging service, a sampling service, a sample detection service, or the like, or any combination thereof. In some embodiments, each of one type of medical service may involve one or more medical procedures. As used herein, a medical procedure may refer to an activity or a series of actions attended to achieve a result in the delivery of healthcare, for example, directed at or performed on a subject (e.g., a patient) to measure, diagnosis and/or treat the subject. Exemplary medical procedures may include a diagnostic procedure (e.g., an imaging procedure, a sampling and examination procedure), a treatment procedure (e.g., a radiotherapy treatment procedure), etc. In some embodiments, the subject may include a body, a substance, or the like, or any combination thereof. In some embodiments, the subject may include a specific portion of a body, such as a head, a thorax, an abdomen, or the like, or any combination thereof. In some embodiments, the subject may include a specific organ, such as an esophagus, a trachea, a bronchus, a stomach, a gallbladder, a small intestine, a colon, a bladder, a ureter, a uterus, a fallopian tube, etc. In some embodiments, the subject may include a physical model (e.g., a water phantom). In the present disclosure, “object” and “subject” are used interchangeably. In some embodiments, the movable medical device 110 may include a couch. The subject may be placed on the couch for receiving a medical service.

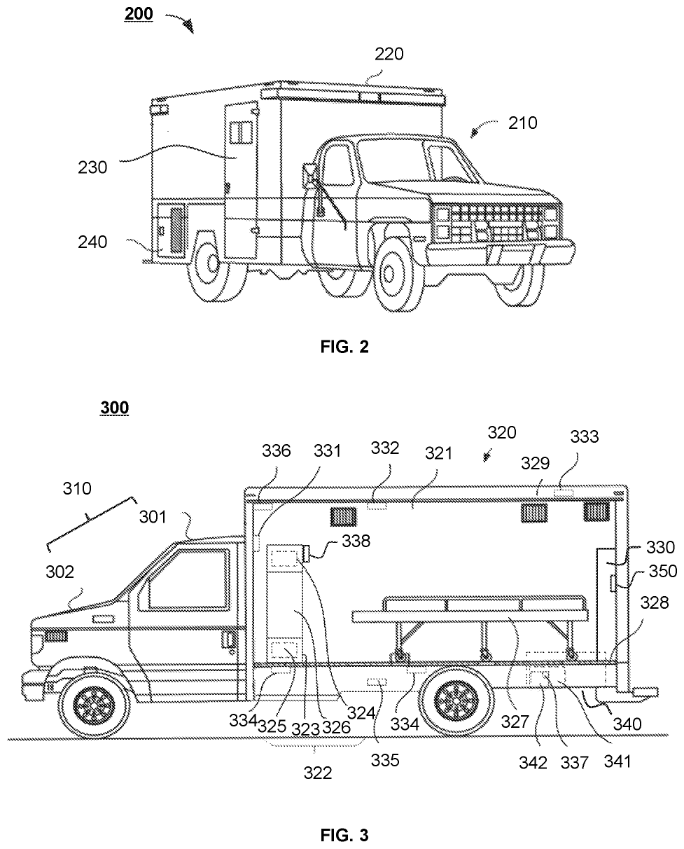

The movable medical device 110 may include one or more imaging devices. The imaging device may be configured to obtain image data including a representation of at least a portion of the movable medical device 110. The image data may include a static image, a video, an image sequence including multiple static images, etc. The movable medical device 110 may be installed within the field of view of the imaging device. For example, the movable medical device 110 and the imaging device may be located in the same room. As another example, the imaging device may be mounted on the ceiling or a wall of the room where the movable medical device 110 is located. The imaging device may include one or more visual sensors, etc. The visual sensors may refer to an apparatus for visual recording. The visual sensors may capture the image data including the representation of at least a portion of the movable medical device 110. In some embodiments, the visual sensors may be located at different positions. The movable medical device 110 may be located within the field view of each of at least a portion of the visual sensors. The visual sensors may capture image data representing different parts of the movable medical device 110 from different perspectives. In some embodiments, the visual sensors may include a stereo camera configured to capture a static image or video. The stereo camera may include a binocular vision device or a multi-camera. In some embodiments, the imaging device may transmit the collected image data to the processing device 120, the storage device 130, and/or the terminal(s) 140 via the network 150. In some embodiments, the medical cabin may include a radiation room, a sample processing room, a control room, or the like, or any combination thereof. The radiation room may be configured to accommodate a radiation device for providing the medical imaging and/or treatment service. In some embodiments, the radiation device may include an imaging device, a treatment device (e.g., radiotherapy equipment), a multi-modality device to acquire one or more images of different modalities or acquire an image relating to at least one part of a subject and perform treatment on the at least one part of the subject, etc. The imaging device may be configured to generate an image including a representation of at least one part of the subject. Exemplary imaging devices may include, for example, a computed tomography (CT) device, a cone beam CT device, a positron emission computed tomography (PET) device, a volume CT device, a magnetic resonance imaging (MRI) device, or the like, or a combination thereof. The treatment device may be configured to perform a treatment on at least one part of the subject. Exemplary treatment devices may include a radiotherapy device (e.g., a linear accelerator), an X-ray treatment device, etc. The sample processing room may be configured to accommodate medical resources for providing the sampling service and/or the sample detection service. The control room may be configured to accommodate a control platform used to control one or more components (e.g., the radiation device) of the movable medical device 110. In some embodiments, the movable platform may be configured to move the medical cabin to a desired location. More descriptions regard the movable medical device 110 may be found elsewhere in the present disclosure (e.g., FIGS. 2-3, FIGS. 9-11, and the descriptions thereof).

In some embodiments, the movable medical device 110 may transmit the image data via the network 150 to the processing device 120, the storage device 130, and/or the terminal(s) 140. For example, the image data may be sent to the processing device 120 for further processing or may be stored in the storage device 130. In some embodiments, the movable medical device 110 may be configured to provide multiple types of medical services in response to a control signal generated by the processing device 120.

The processing device 120 may process data and/or information obtained from the movable medical device 110, the storage device 130, and/or the terminal(s) 140. For example, the processing device 120 may obtain a monitoring result related to at least one of an operation state of at least one of components of the movable medical system or an environment parameter where the medical cabin is located acquired by a monitoring device; and provide feedback based on the monitoring result. The feedback may include a prompt instruction associated with the monitoring result, an adjustment instruction for adjusting an operation of one or more components related to the monitoring result, or the like, or any combination thereof. As another example, the processing device 120 may control the operation of one or more medical resources for providing multiple types of medical services in the movable medical device 110 based on data and/or information obtained from the movable medical device 110, the storage device 130, and/or the terminal(s) 140. In some embodiments, the processing device 120 may be a single server or a server group. The server group may be centralized or distributed. In some embodiments, the processing device 120 may be local or remote. For example, the processing device 120 may be integrated into the control platform (or a control device) located in the medical cabin and access information and/or data from the movable medical device 110, the storage device 130, and/or the terminal(s) 140 via the network 150. As another example, the processing device 120 may be directly connected to the movable medical device 110, the terminal(s) 140, and/or the storage device 130 to access information and/or data. In some embodiments, the processing device 120 may be implemented on a cloud platform. For example, the cloud platform may include a private cloud, a public cloud, a hybrid cloud, a community cloud, a distributed cloud, an inter-cloud, a multi-cloud, or the like, or a combination thereof. In some embodiments, the processing device 120 may be part of the terminal 140. In some embodiments, the processing device 120 may be part of the movable medical device 110. For example, the processing device 120 may be a part of the control device in the monitoring system, the power supply system, etc.

The storage device 130 may store data, instructions, and/or any other information. In some embodiments, the storage device 130 may store data obtained from the movable medical device 110, the processing device 120, and/or the terminal(s) 140. The data may include image data acquired by the processing device 120, algorithms and/or models for processing the image data, etc. In some embodiments, the storage device 130 may store data and/or instructions that the processing device 120 and/or the terminal 140 may execute or use to perform exemplary methods described in the present disclosure. In some embodiments, the storage device 130 may include mass storage, removable storage, a volatile read-and-write memory, a read-only memory (ROM), or the like, or any combination thereof. Exemplary mass storage may include a magnetic disk, an optical disk, a solid-state drive, etc. In some embodiments, the storage device 130 may be implemented on a cloud platform.

In some embodiments, the storage device 130 may be connected to the network 150 to communicate with one or more other components in the movable medical system 100 (e.g., the processing device 120, the terminal(s) 140). One or more components in the movable medical system 100 may access the data or instructions stored in the storage device 130 via the network 150. In some embodiments, the storage device 130 may be integrated into the movable medical device 110.

The terminal(s) 140 may be connected to and/or communicate with the movable medical device 110, the processing device 120, and/or the storage device 130. In some embodiments, the terminal 140 may include a mobile device 141, a tablet computer 142, a laptop computer 143, or the like, or any combination thereof. For example, the mobile device 141 may include a mobile phone, a personal digital assistant (PDA), a gaming device, a navigation device, a point of sale (POS) device, a laptop, a tablet computer, a desktop, or the like, or any combination thereof. In some embodiments, the terminal 140 may include an input device, an output device, etc. The input device may include alphanumeric and other keys that may be input via a keyboard, a touchscreen (for example, with haptics or tactile feedback), a speech input, an eye tracking input, a brain monitoring system, or any other comparable input mechanism. Other types of the input device may include a cursor control device, such as a mouse, a trackball, or cursor direction keys, etc. The output device may include a display, a printer, or the like, or any combination thereof.

The network 150 may include any suitable network that can facilitate the exchange of information and/or data for the movable medical system 100. In some embodiments, one or more components of the movable medical system 100 (e.g., the movable medical device 110, the processing device 120, the storage device 130, the terminal(s) 140, etc.) may communicate information and/or data with one or more other components of the movable medical system 100 via the network 150. The network 150 may be and/or include a public network (e.g., the Internet), a private network (e.g., a local area network (LAN), a wide area network (WAN)), etc.), a wired network (e.g., an Ethernet network), a wireless network (e.g., an 802.11 network, a Wi-Fi network, etc.), a cellular network (e.g., a Long Term Evolution (LTE) network), a frame relay network, a virtual private network (VPN), a satellite network, a telephone network, routers, hubs, witches, server computers, and/or any combination thereof. In some embodiments, the network 150 may include one or more network access points. For example, the network 150 may include wired and/or wireless network access points such as base stations and/or internet exchange points through which one or more components of the movable medical system 100 may be connected to the network 150 to exchange data and/or information.

This description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. However, those variations and modifications do not depart the scope of the present disclosure. In some embodiments, the movable medical system 100 may include one or more additional components and/or one or more components described above may be omitted. Additionally or alternatively, two or more components of the movable medical system 100 may be integrated into a single component. For example, the processing device 120 may be integrated into the movable medical device 110. As another example, a component of the movable medical system 100 may be replaced by another component that can implement the functions of the component.

FIG. 2 is a schematic diagram illustrating an exemplary movable medical device 200 according to some embodiments of the present disclosure. In some embodiments, the movable medical device 200 may be an exemplary embodiment of the movable medical device 110 as described in FIG. 1.

As shown in FIG. 2, the movable medical device 200 may include a movable platform 210 and a medical cabin 220.

The movable platform 210 may be configured to move the medical cabin 220 to a desired location. The desired location may refer to a destination location, such as a street, a market, a residential district, or any location where needs the movable medical device 200. For example, the desired location may be a location where an infectious disease (e.g., a flu SARS-CoV-2) appears or gathers. Exemplary movable platforms 210 may include a van, a truck, a multi-purpose vehicle, a ship, an aircraft, etc. In some embodiments, the movable platform 210 may include a main body and a support portion connected to the main body. The main body may be configured to accommodate a movement controller, a driving component, an electric generator, etc. The movement controller may be configured to control a motion (e.g., a direction, a speed, etc.) of the movable medical device 200. Exemplary movement controller may include a steering wheel, a rudder, a steering shaft, or the like, or any combination thereof. The driving component may be configured to drive the movable platform to move by, e.g., providing a power source for the movable medical device 200. The driving component may include an internal combustion engine (ICE), an electric motor, or a combination thereof. The ICE may refer to a heat engine in which the combustion of a fuel (e.g., petrol, diesel, propane, natural gas, etc.) occurs with an oxidizer (usually air) in a combustion chamber of the ICE. In the ICE, the expansion of high-temperature and high-pressure gases produced by combustion may apply direct force to one or more components of the ICE to drive the movable medical device 200 to move. The electric motor may use energy stored in batteries to provide force to one or more components of the electric motor to drive the movable medical device 200 to move. In some embodiments, the driving component may include the ICE and the electric motor. The movable medical device 200 may be driven to move by the ICE and/or the electric motor. In some embodiments, the movable platform 210 may further include a power generation component. The power generation component may be configured to provide power for one or more devices (e.g., an air conditioner, a speaker, a displayer, etc.) in the movable platform 210. In some embodiments, the power generation component may include an alternating current (AC) power generation component, a direct current (DC) power generation component, or a combination thereof. When a DC power generation component is used, a transducer may be disposed in the circuit to transfer a direct current to an alternating current. More descriptions regarding the movable platform 210 may be found elsewhere in the present disclosure (e.g., FIGS. 3, 9-11, and the descriptions thereof).

The medical cabin 220 may be configured to accommodate medical resources for providing one or more types of medical services. For example, the medical cabin 220 may include a radiation room, a sample processing room, a control room, or the like, or any combination thereof. The radiation room may be configured to accommodate medical sources (e.g., a radiation device, a table, etc.) for providing one or more medical services associated with radiation (e.g., an X-rays radiation imaging procedure, a radiotherapy treatment procedure, etc). In some embodiments, the radiation device may be a medical device, for example, a positron emission tomography (PET) device, a single-photon emission computed tomography (SPECT) device, a computed tomography (CT) device, a magnetic resonance imaging (MRI) device, a radiation therapy (RT) device, or the like, or any combination thereof. The sample processing room may be configured to accommodate medical resources for providing the sampling service and/or the sample detection service. Exemplary samples may include a throat swab sample, a nasopharyngeal swab sample, an oropharyngeal swab sample, a sample of a bronchoalveolar lavage fluid, a biopsy specimen of a lung, a sputum specimen, a blood sample, or the like, or any combination thereof. The control room may be configured to accommodate a control platform used to control operation of medical resources (e.g., the radiation device) for providing the medical services. In some embodiments, the radiation room may be disposed of independently of the sample processing room, the control room, etc. For example, the radiation room may be isolated by protection materials, to prevent radiation generated by the radiation device (e.g., the CT device) in the radiation room from leaking to other components (e.g., the sample processing room, the control room, etc.) of the movable medical device 200. More descriptions regarding the medical cabin 220 may be found elsewhere in the present disclosure (e.g., FIGS. 3, 9-11, and the descriptions thereof).

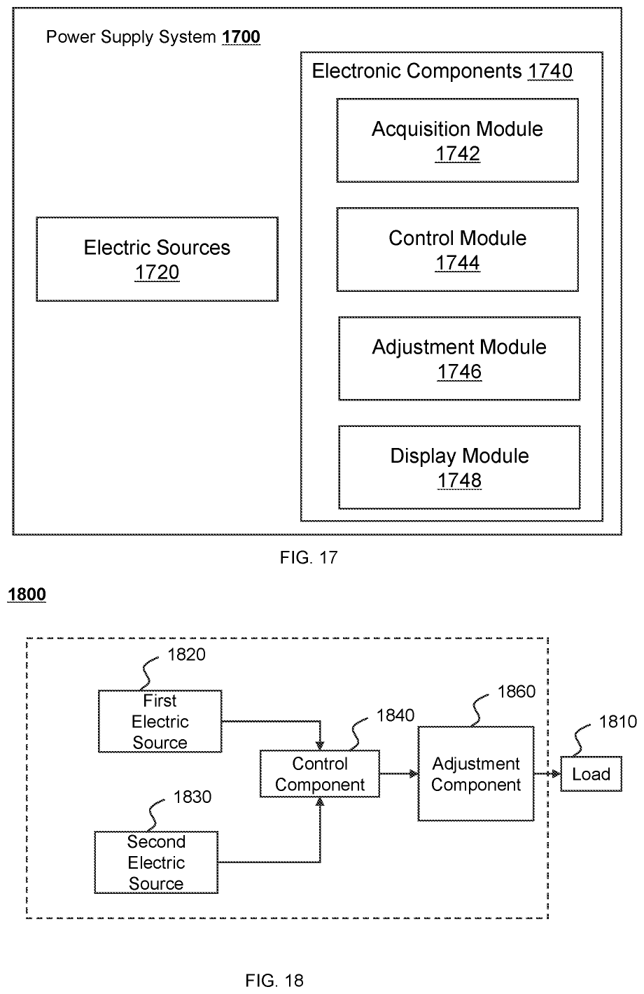

In some embodiments, the movable medical device 200 may include a power supply system. The power supply system may be configured to provide power for one or more components of the movable medical device 200. In some embodiments, the power supply system may include a driving assembly (e.g., the driving component as described above), an electric supply assembly, etc. The driving assembly may be configured to provide power for the movement of the movable medical device 200. The driving assembly may include a driving component. The driving component may include an actuator, such as an electric actuator, an ICE, or a combination thereof. In some embodiments, the driving component may be disposed at a front portion and/or a rear portion of the movable medical device 200. The electric supply assembly may be configured to provide electric power for one or more components of the movable medical device 200. The electric supply assembly may include a power generation component as described above, a power storage component, etc. The electric supply assembly may be disposed at a front portion and/or a rear portion of the movable medical device 200. For example, the electric supply assembly may provide electric power for one or more loads in the movable medical device, such as air conditioning, a loudspeaker box, and other loads in the movable platform 210 of the movable medical device 200. As another example, when the driving component includes an electric actuator, the electric supply assembly may provide electric power for the electric actuator. As still another example, the electric supply assembly may provide electric power for a medical device in the cabin of the movable medical device 200, such as a radiation device. More descriptions regarding the electric supply assembly may be found elsewhere in the present disclosure (e.g., FIGS. 17-21 and the descriptions thereof). In some embodiments, the medical cabin 220 may include an electric room 240. The electrical room 240 may be configured to accommodate one or more components of the power supply system. In some embodiments, the power device may include an energy storage component and/or an electric generator. The energy storage component may be configured to supply electrical energy to the movable platform 210 and/or the medical cabin 220. The electric generator may be configured to power the energy storage component. The electric room 240 may be disposed under the rear portion of the movable medical device 200. Therefore, the electric room 240 may be away from the radiation device, so that influences on the radiation device caused by the power device in the electric room240 may be reduced. For example, the energy storage component and/or the electric generator may generate noise, vibration, etc., during operation. The electric room 240 may be disposed outside the medical cabin 220 to reduce influences of noise or vibration on the radiation device. More descriptions regarding the power supply system may be found elsewhere in the present disclosure (e.g., FIGS. 3, 17-21, and the descriptions thereof).

In some embodiments, the movable medical device 200 may include a monitoring system 230. The monitoring system 230 may be configured to monitor an operation of one or more components in the movable medical device 200 and/or an operation environment of the movable medical device 200. In some embodiments, the monitoring system 230 may include one or more monitoring devices and a control device. The one or more monitoring devices may be configured to acquire a monitoring result related to at least one of an operation state of at least one of components of the movable medical device 200 or an environment parameter where the medical cabin 220 is located. The control device may be configured to provide feedback based on the monitoring result. In some embodiments, the monitoring devices may be disposed at a front portion of the movable medical device 200, a rear portion of the movable medical device 200, or any suitable positions outside the movable medical device 200. In some embodiments, the control device of the monitoring system may be integrated into the control platform in the control room. In some embodiments, the control device of the monitoring system may be separated from the control platform in the control room. More descriptions regarding the monitoring system 230 may be found elsewhere in the present disclosure (e.g., FIGS. 3-6 and the descriptions thereof).

The description is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the one or more monitoring devices and the control device may be located in different spaces. However, those variations and modifications do not depart from the scope of the present disclosure.

FIG. 3 is a schematic diagram illustrating an exemplary movable medical device 300 according to some embodiments of the present disclosure. In some embodiments, the movable medical device 300 may be an exemplary embodiment of the movable medical device 110 as described in FIG. 1 or the movable medical device 200 as described in FIG. 2. The following descriptions are provided regarding a movable CT device as the movable medical device 300 unless otherwise stated. It should be noted that the descriptions of the movable CT system in the present disclosure are merely provided for illustration, and not intended to limit the scope of the present disclosure.

As shown in FIG. 3, the movable medical device 300 may include a movable platform 310, a medical cabin 320, a monitoring system 330, and a power supply system 340.

The movable platform 310 may be configured to move the medical cabin 320 to a desired location. In some embodiments, the movable platform 310 may be implemented by a vehicle. The vehicle may include a vehicle body and a carriage connected to the vehicle body. The vehicle body may serve as the movable platform 310 of the movable medical device 300, and the carriage may serve as the medical cabin 320 of the movable medical device 300. The vehicle body may include a vehicle head (i.e., the main body of the movable platform 310) and a chassis (i.e., the support portion of the movable platform 310) configured to support the carriage. The vehicle may also include a power assembly (e.g., an engine, an alternator, and a gearbox, etc.) configured to provide power for the movement of the vehicle, an electronic and electrical system, and other accessories mounted on the vehicle head and/or the chassis. The vehicle head may include a cab 301 and an engine compartment 302 arranged in front of the cab 301. The power assembly (e.g., the engine) may be provided in the engine compartment 302 and provide a power source for the vehicle. For example, the alternator may be set in the engine compartment 302 to provide electric energy for one or more loads in the vehicle, such as air conditioning, a loudspeaker box, and other loads in the cab 301.

The medical cabin 320 may be configured to accommodate medical resources for providing one or more types of medical services. For example, the medical cabin 320 may include a radiation room 321, a sample processing room (not shown), a control room (not shown), or the like, or any combination thereof. The radiation room 321 may be configured to accommodate a radiation device 322 for providing a medical imaging service. In some embodiments, the radiation room 321 may be surrounded by one or more protection materials 329 to prevent radiation generated by the radiation device 322 in the radiation room from leaking to other components (e.g., the sample processing room, the control room, etc.) of the movable medical device 300. Exemplary protection materials 329 may include heavy metal materials (e.g., lead, steel), concrete (e.g., radiation-proof concrete, ordinary concrete, etc.), clay brick, compacted soil, graphite, radiation-proof glass, radiation-proof rubber, radiation-proof plastic, or the like, or any combination thereof. In some embodiments, a couch 327 may be located in the radiation room 321. The radiation device 322 and the couch 327 may be fixed on a floor 328 in the medical cabin 320 through a rigid material (e.g., a steel plate, a rigid cushion). The fixing manner may include a bolted connection, a screwing connection, a welding connection, a riveting connection, an interference-fit connection, or the like, or any combination thereof. In some embodiments, the couch 327 may be movable on the floor 328 in the medical cabin 320 and the radiation device 322 may be fixed on the floor 328, so that the couch 327 may be caused to move toward the radiation device 322 to a designated position for scanning. For example, the couch 327 may be provided with a rigid material (e.g., a steel plate, a rigid cushion) or through a movable structure. The movable structure may include a pulley, a guide rail, a transmission chain, or the like, or any combination thereof. In some embodiments, the radiation device 322 may be movably disposed on the floor 328 in the medical cabin 320 and the couch 327 may be fixed on the floor 328. Alternatively, the radiation device 322 and the couch 327 may be both movable on the floor 328, so that a distance between the radiation device 322 and the couch 327 may be controlled based on scanning requirements.

Merely by way of example, the radiation device 322 may be a CT device. The CT device may include an annular or substantially annular structure. In some embodiments, the CT device may include a gantry 323, a radiation source 324, and a detector 325. The radiation source 324 and the detector 325 may be symmetrically disposed along a diameter of the gantry 323. Radiation rays emitted by the radiation source 324 may be detected by the detector 325 to scan a subject. The subject may include a body, a substance, or the like, or any combination thereof. More descriptions regarding the subject may be found elsewhere in the present disclosure (e.g., FIG. 1 and the descriptions thereof). A bore 326 may be located in the gantry 323. The bore 326 may be configured to allow at least a portion of the couch 327 to enter the gantry 323. Therefore, the radiation device 322 may be configured to scan a target portion of the subject (e.g., a region of interest) on the couch 327. It should be noted that the descriptions of the CT device as shown in FIG. 2 are merely provided for illustration, and not intended to limit the scope of the present disclosure. For example, the movable medical device 300 may include other medical devices, such as the PET device, the SPECT device, the MRI device, the RT device, etc.

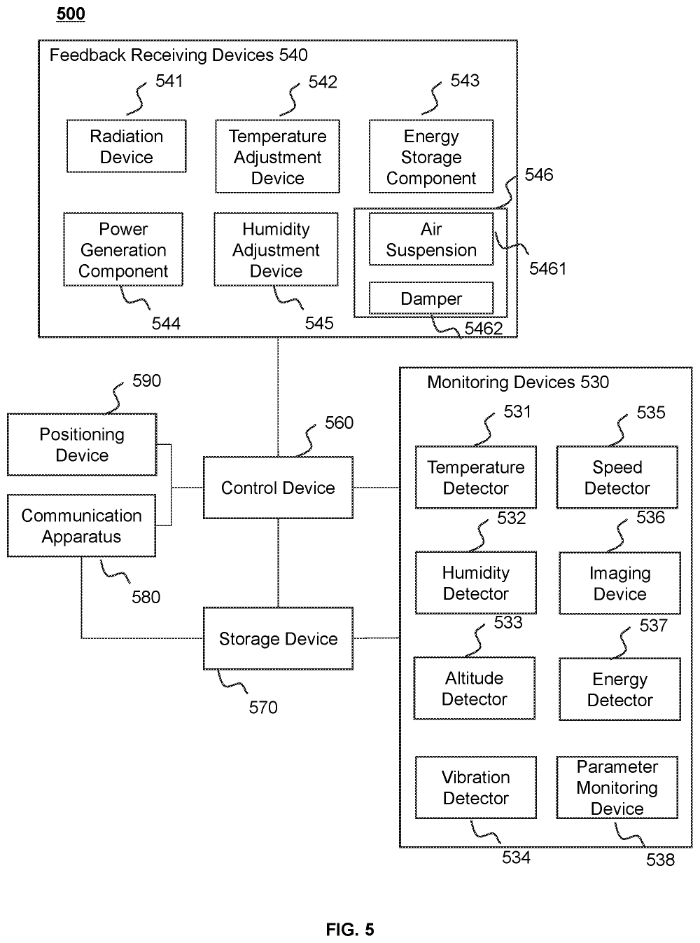

The monitoring system may include one or more monitoring devices and a control device 350. The one or more monitoring devices may be configured to acquire a monitoring result related to at least one of an operation state of at least one of components of the movable medical device 300 or an environment parameter where the medical cabin 320 is located. The control device 350 may be configured to provide feedback based on the monitoring result. The feedback may include a prompt instruction associated with the monitoring result, an adjustment instruction for adjusting or controlling an operation of one or more components related to the monitoring result and/or the environment parameter, or the like, or any combination thereof. In some embodiments, the one or more monitoring devices may include a temperature detector 331, a humidity detector 332, an altitude detector 333, a vibration detector 334, a speed detector 335, an imaging device 336, an energy detector 337, a state monitoring device 338, or the like, or any combination thereof. More descriptions regarding the one or more monitoring devices may be found elsewhere in the present disclosure (e.g., FIG. 5 and the descriptions thereof).

The power supply system 340 may be configured to accommodate a power generation component 341 and/or an energy storage component 342. The power generation component 341 and/or the energy storage component 342 may be configured to supply power to one or more loads in the movable medical device 300. In some embodiments, the energy storage component 342 may be powered by the power generation component 341. The power generation component 341 may include an AC power generation component, a DC power generation component, or a combination thereof. When a DC power generation component is used, a transducer may be located in the circuit to transfer a direct current to an alternating current. In some embodiments, a type of the power generation component 341 may be the same as or different from a type of the power generation component in the movable platform 310. For example, the power generation component 341 and the power generation component in the movable platform 310 may be the AC power generation components. As another example, the power generation component 341 may be the AC power generation component, and the power generation component in the movable platform 310 may be the DC power generation component. The energy storage component 342 may include a battery, a rechargeable battery, etc., of an uninterrupted power supply (UPS). The energy storage component 342 may be configured to supply power to the radiation device 322. In some embodiments, no energy storage component may be disposed. The power generation component 341 may be configured to supply power to the radiation device 322. More descriptions regarding the power generation component and/or the energy storage component may be found elsewhere in the present disclosure (e.g., FIGS. 17-21 and the descriptions thereof).

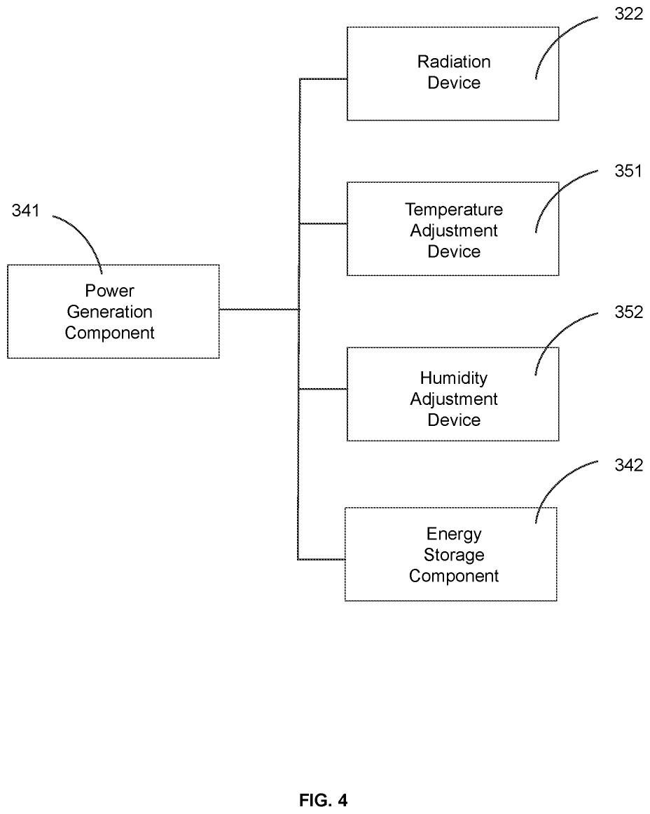

The one or more loads may be electrically connected with the power generation component 341 and/or the energy storage component 342. Referring to FIG. 4, FIG. 4 is a schematic diagram illustrating an exemplary electrical connection according to some embodiments of the present disclosure. As shown in FIG. 4, the power generation component 341 may be electrically connected with the radiation device 322, a temperature adjustment device 351, a humidity adjustment device 352, and the energy storage component 342, respectively. It should be noted that the descriptions of the electrical connection as shown in FIG. 4 are merely provided for illustration, and not intended to limit the scope of the present disclosure. For example, the power generation component 341 may be electrically connected with other loads, such as lighting, other temperature adjustment devices, other humidity adjustment devices, etc.

In some embodiments, the radiation device 322, the power generation component 341, the energy storage component 342, etc., may be affected by the environment, such as temperature, humidity, altitude, vibration, etc. For example, the radiation device 322 may be sensitive to temperature and humidity. If one of the temperature and humidity exceeds a predetermined range, the radiation device 322 may be affected. The predetermined range may refer to a maximum temperature or a maximum humidity where the radiation device 322 may be normally operated without damage or influence for the radiations generated by the radiation device 322. The predetermined range may be determined based on a temperature test or a humidity test on the radiation device 322. As another example, operation powers of the power generation component 341, the energy storage component 342, the air conditioner, etc., may be affected by the altitude. Therefore, the one or more monitoring devices of the monitoring system 330 may acquire the monitoring result related to at least one of an operation state of at least one of components of the movable medical device or an environment parameter where the medical cabin is located. The control device 360 of the monitoring system 330 may be configured to provide the feedback based on the monitoring result. One or more components (e.g., the temperature adjustment device 351, the humidity adjustment device 352, etc.) of the movable medical device 300 may be caused to adjust the at least one of an operation parameter of at least one of components of the movable medical device or an environment parameter where the medical cabin 320 is located based on the feedback. More descriptions regarding the adjustment may be found elsewhere in the present disclosure (e.g., FIG. 5 and the descriptions thereof).

FIG. 5 is a block diagram illustrating an exemplary monitoring system of a movable medical device according to some embodiments of the present disclosure. In some embodiments, the movable medical device in FIG. 5 may be an example of the movable medical device 110 described in FIG. 1, the movable medical device 200 described in FIG. 2, the movable medical device 300 as described in FIG. 3.

The monitoring system 500 may include one or more monitoring devices 530 may be configured to acquire a monitoring result related to at least one of an operation state of at least one of components of the movable medical device or an environment parameter (e.g., a temperature, a humidity, an altitude, a vibration, etc.) where the medical cabin is located. As shown in FIG. 5, the one or more monitoring devices 530 may include a temperature detector 531, a humidity detector 532, an altitude detector 533, a vibration detector 534, a speed detector 535, an imaging device 536, an energy detector 537, and a state monitoring device 538.

The temperature detector 531 may be configured to detect a temperature inside and/or outside the medical cabin. Exemplary temperature detectors may include a thermocouple, a thermistor, a resistance temperature detector (RTD), a pyrometer, an infrared thermometer, or the like, or any combination thereof. In some embodiments, the temperature detector 531 may be disposed on a position inside the medical cabin, such as an inner sidewall, an inner top wall, an inner bottom wall, etc., of a medical cabin (e.g., the medical cabin 320 as described in FIG. 3) of the movable medical system, so that the temperature detector 531 may detect the temperature inside the medical cabin. In some embodiments, the temperature detector 531 may be disposed on a position outside the medical cabin, such as an outer sidewall, an outer top wall, an outer bottom wall, etc. of the medical cabin, so that the temperature detector 531 may detect the temperature outside the medical cabin.

The humidity detector 532 may be configured to detect a humidity inside and/or outside the medical cabin. Exemplary humidity detectors may include a capacitive humidity detector, a resistive humidity detector, a thermal humidity detector, a gravimetric humidity detector, an optical humidity detector, or the like, or any combination thereof. In some embodiments, the humidity detector 532 may be disposed on a position inside the medical cabin, such as an inner sidewall, an inner top wall, an inner bottom wall, etc., of a medical cabin (e.g., the medical cabin 320 as described in FIG. 3) of the movable medical system, so that the humidity detector 532 may detect the humidity inside the medical cabin. In some embodiments, the humidity detector 532 may be disposed on a position outside the medical cabin, such as an outer sidewall, an outer top wall, an outer bottom wall, etc. of the medical cabin, so that the humidity detector 532 may detect the humidity outside the medical cabin.

The altitude detector 533 may be configured to detect an altitude where the medical cabin (e.g., the medical cabin 320 as described in FIG. 3) of the movable medical system is located. The altitude detector 533 may be an altimeter, such as a pressure altimeter, a sonic altimeter, a radar altimeter, a global positioning system (GPS), etc. In some embodiments, the altitude detector 533 may be disposed on a position inside/outside the medical cabin, such as an inner sidewall, an inner top wall, an inner bottom wall, an outer sidewall, an outer top wall, an outer bottom wall, etc., of a medical cabin (e.g., the medical cabin 320 as described in FIG. 3) of the movable medical system, so that the altitude detector 533 may detect the altitude of the medical cabin. In some embodiments, the medical cabin may be disposed no altitude detector, and the altitude where the medical cabin is located may be determined based on the temperature, the humidity, air pressure, etc., outside the medical cabin.

The vibration detector 534 may be configured to detect vibrations (e.g., an intensity of the vibrations (i.e., the vibration amplitude), a vibration acceleration, a vibration frequency) of the medical cabin. Exemplary vibration detectors may include a sensor (e.g., an accelerometer, an associated microelectromechanical system), a spring-mass system, a magnetic ball, or the like, or any combination thereof. In some embodiments, the vibration detector 534 may be disposed on a position outside the medical cabin, such as an outer sidewall, an outer top wall, an outer bottom wall, etc. of a medical cabin (e.g., the medical cabin 320 as described in FIG. 3) of the movable medical system, so that the vibration detector 334 may detect the vibrations outside the medical cabin.

The speed detector 535 may be disposed at a suitable position of the medical cabin, such as an inner side or an outer sidewall of a medical cabin (e.g., the medical cabin 320 as described in FIG. 3) of the movable medical system, so that the speed detector 535 may detect parameters (e.g., speed, acceleration) related to the speed during the movement. Exemplary speed detectors may include a radar detector, an optical detector, etc. In some embodiments, the parameters related to the speed may be determined combined with a dashboard in the movable platform (e.g., the movable platform 310 as described in FIG. 3) of the movable medical system.

In some embodiments, the imaging device 536 may include one or more first imaging devices and/or one or more second imaging devices. The one or more first imaging devices may be disposed on a position inside the medical cabin, such as an inner sidewall, an inner top wall, an inner bottom wall, etc. of a medical cabin (e.g., the movable platform 310 as described in FIG. 3) of the movable medical system, so that the one or more first imaging devices may be configured to acquire a monitoring result including image data of a scene inside the medical cabin. The monitoring result including the image data of the scene inside the medical cabin may indicate a state of at least one of the service receiver, the service provider, and/or one of one or more components in the medical cabin. The one or more second imaging devices may be disposed on a position outside the medical cabin, such as an outer sidewall, an outer top wall, an outer bottom wall, etc. of the medical cabin (e.g., the movable platform 310 as described in FIG. 3) of the movable medical system, so that the one or more second imaging devices may be configured to acquire a monitoring result including image data of a scene outside the medical cabin. The monitoring result including the image data of the scene outside the medical cabin may indicate a state of the environment outside the medical cabin, such as a road (e.g., flatness, congestion, etc., of the road). In some embodiments, the imaging device 536 may include a visual imaging device, e.g., a camera, a video recorder, an image sensor, etc. Exemplary cameras may include a gun camera, a dome camera, an integrated camera, a monocular camera, a binocular camera, a multi-view camera, or the like, or any combination thereof. Exemplary video recorders may include a personal computer (PC) digital video recorder (DVR), an embedded DVR, a visible light DVR, or the like, or any combination thereof. Exemplary image sensors may include a charge-coupled device (CCD) image sensor, a complementary metal-oxide semiconductor (CMOS) image sensor, or the like, or any combination thereof. In some embodiments, the imaging device 536 may include a thermal imaging device, e.g., a thermal imaging camera, a thermal imaging DVR, or the like, or any combination thereof. The thermal imaging device may include a thermal imaging device with a cooled infrared image detector and a thermal imaging device with an uncooled detector.

The energy detector 537 may be configured to determine a remain energy (or capacity) of an energy storage component (e.g., the energy storage component 342, an energy storage component 543, etc.) of the movable medical device and/or a current volume of fuel in a fuel tank of the power generation component (e.g., the power generation component 341, a power generation component 544, etc.). Exemplary energy detectors may include a battery management system (BMS), a fuel gauge, etc. In some embodiments, the energy detector 537 may be located in the fuel tank of the power generation component (e.g., the power generation component 341, the power generation component 544, etc.). In some embodiments, the energy detector 537 may be electrically connected with the energy storage component (e.g., the energy storage component 342, an energy storage component 543, etc.).

The state monitoring device 538 may be configured to monitor an operation state of the radiation device (e.g., the radiation device 322, a radiation device 541). Exemplary state monitoring devices may include a vibration sensor, a plurality of detecting units that receive radiation rays, etc. The state monitoring device 538 may be located on the radiation device 541. The operation state of the radiation device 322 may include a normal operation, an anomaly, etc. Exemplary anomalies may include a malfunction of a tube (e.g., a filament of the tube, an anode target, a high-voltage generator), a malfunction of a collimator, a malfunction of a detector, etc.

In some embodiments, the monitoring devices 530 may include an air quality detector (not shown in FIG. 5), such as an air monitor. The air quality detector may be configured to detect air quality indexes such as indexes related to gases (e.g., ammonia, carbon monoxide, sulfur dioxide, nitrous oxides, methane, carbon dioxide, and chlorofluorocarbons), particulates (both organic and inorganic), biological molecules, etc.

In some embodiments, two or more monitoring devices among the one or more monitoring devices may be integrated into a monitoring device. The integrated monitoring devices may implement two or more functions. For example, a temperature detector and a humidity detector may be integrated into a temperature and humidity detector for detecting the temperature and humidity inside/outside the medical cabin.

The monitoring system 500 may include a control device 560. The control device 560 may be configured to provide feedback based on the monitoring result acquired by at least one of the monitoring devices 530. The feedback may include a prompt instruction associated with the monitoring result, an adjustment instruction for adjusting an operation of one or more components related to the monitoring result, or the like, or any combinations thereof. The one or more components related to the monitoring result may also be referred to as feedback receiving components 540. As shown in FIG. 5, the feedback receiving devices 540 may include a radiation device 541, a power generation component 544, an energy storage component 543, a temperature adjustment device 542, a humidity adjustment device 545, a damping apparatus 546, etc. The feedback receiving devices 540 may be adjusted according to the received feedback.

The radiation device 541 may be a medical device, for example, a positron emission tomography (PET) device, a single-photon emission computed tomography (SPECT) device, a computed tomography (CT) device, a magnetic resonance imaging (MRI) device, a radiation therapy (RT) device, or the like, or any combination thereof. The power generation component 544 and/or the energy storage component 543 may be configured to generate power for a load in the movable medical device. The temperature adjustment device 542 may be configured to adjust the temperature in the medical cabin. The humidity adjustment device 545 may be configured to adjust the humidity in the medical cabin. The damping apparatus 546 may be configured to reduce the vibration of the medical cabin.

The control device 560 may be communicated with the one or more monitoring devices 530. The monitoring result acquired by the one or more monitoring devices 530 may be transmitted to the control device 560 through a wired or wireless connection (e.g., the communication apparatus 580). For example, the one or more monitoring devices may be connected with the control device 560 through a pre-embedded cable. The monitoring result may be transmitted to the control device 560 through the cable. As another example, the one or more monitoring devices may be connected with the control device 360 through a cable and a trunking. Disposing the cable through the trunking may facilitate the maintenance of the one or more monitoring devices in the later period. In addition, the cable may be replaced easily when the cable is aging or damaged. As still another example, the data acquired by the one or more monitoring devices may be transmitted to the control device 560 through Bluetooth or WiFi.

In some embodiments, the control device 560 may provide the feedback based on the monitoring result. For example, after the control device 560 obtains the monitoring result, the control device 560 may determine the feedback by determining whether the monitoring result satisfies one or more abnormal conditions. In some embodiments, in response to determining that the monitoring result satisfies one or more abnormal conditions, the control device 560 may determine the feedback indicating that the monitoring result satisfies the one or more abnormal conditions; in response to determining that the monitoring result does not satisfy the one or more abnormal conditions, the control device 560 may determine the feedback indicating that the monitoring result does not satisfy the one or more abnormal conditions or satisfies one or more normal conditions. In some embodiments, in response to determining that the monitoring result satisfies one or more abnormal conditions, the control device 560 may determine the feedback indicating that the monitoring result satisfies the one or more abnormal conditions; in response to determining that the monitoring result does not satisfy the one or more abnormal conditions, the control device 560 may not provide feedback. The one or more conditions may be that the remain volume of the fuel is lower than a fuel threshold, the temperature in the medical cabin is lower or higher than a temperature threshold, the humidity in the medical cabin is lower or higher than a humidity threshold, a vibration acceleration reaches a vibration threshold, etc.

In some embodiments, the control device 560 may be configured to cause the feedback receiving devices 540 to adjust the operation state of the feedback receiving devices 540 and/or the environment parameter where the medical cabin is located according to the feedback.

In some embodiments, the control device 560 may determine whether the remaining volume of the fuel in the fuel tank is lower than the volume threshold based on the current volume of the fuel in the fuel tank of the power generation component 544 monitored by the energy detector 537. If the current volume of the fuel is lower than the volume threshold, the control device 560 may generate and output the feedback including a prompt instruction indicating prompt information related to the fuel shortage, a prompt instruction to turn off the power generation component 544, etc. The feedback may be transmitted to the control device 560 for displaying or performing. The volume threshold may be a threshold that can maintain an operation of the power generation component 544 for a period (e.g., 5 minutes, 10 minutes, 15 minutes, 20 minutes, etc.). In some embodiments, the volume threshold may be different or change for multiple different periods. For example, when the remaining volume of the fuel maintains the operation of the power generation component 544 for 20 minutes, the control device 560 may determine that the remaining volume is lower than a first volume threshold. The control device 560 may output prompt information that the remaining volume of the fuel can maintain the operation of the power generation component 544 for 20 minutes. When the remaining volume of the fuel maintains the operation of the power generation component 544 for 10 minutes, the control device 560 may determine that the remaining volume is lower than a second volume threshold. The control device 560 may output prompt information that the remaining volume of the fuel can maintain the operation of the power generation component 544 for 10 minutes.

In some embodiments, the control device 560 may provide the feedback based on the altitude where the medical cabin is located monitored by the altitude detector 533, the influence of different altitudes on the power generation component 544 and/or the energy storage component 543, and the remaining volume of the fuel obtained from the storage device 570 or the energy detector 537. The influence of different altitudes on the power generation component 544 and/or the energy storage component 543 may indicate that a fuel combination, etc., of one of the power generation component 544 and the energy storage component 543 may be increased with an increase of the altitude. For instance, a corresponding relationship between fuel combinations of one of the power generation component 544 and the energy storage component 543 and a plurality of altitudes may be stored in a storage. The control device 560 may retrieve a fuel combination of one of the power generation component 544 and the energy storage component 543 corresponding to an altitude based on the corresponding relationship. For example, an available time (endurance time) of the power generation component 544 may be determined according to the remaining volume of the fuel and the fuel combination corresponding to the altitude. When the available time reaches a predetermined threshold, the control device 560 may prompt to turn off the power generation component 544, automatically turn off the power generation component 544, or turn off a device powered by the power generation component 544. In some embodiments, the control device 560 may automatically calculate the available time of the remaining volume of the fuel according to the power consumption of each radiation of the radiation device 541. The available time may be calculated based on historical power consumption of the radiation device 544 at the altitude. The historical power consumption may include a power consumption corresponding to different body types and different environmental parameters. In some embodiments, the historical power consumption may be stored in the storage device 570, and the control device 560 may retrieve the historical power consumption from the storage device 570.

In some embodiments, the control device 560 may obtain the temperature and/or humidity inside/outside the medical cabin from the temperature detector 531 and/or the humidity detector 532 or the storage device 570. The control device 560 may provide the feedback to adjust the temperature adjustment device 552 and/or the humidity adjustment device 545. For example, the control device 560 may generate a prompt instruction to remind a user to adjust the temperature adjustment device 542 and/or the humidity adjustment device 545. As another example, the control device 560 may generate an adjustment instruction for controlling the temperature adjustment device 542 and/or the humidity adjustment device 545 to adjusting the temperature and/or humidity inside/outside the medical cabin. In some embodiments, the temperature adjustment device 542 may be configured to adjust the temperature in the medical cabin according to the feedback of the control device. Exemplary temperature adjustment devices may include an air conditioner, a heater, etc. The humidity adjustment device 545 may be configured to adjust the humidity in the medical cabin according to the feedback of the control device. Exemplary humidity adjustment devices may include a humidifier, a dehumidifier, etc. For example, if the temperature in the medical cabin is higher than a first temperature threshold, the temperature adjustment device 542 may lower the temperature in the medical cabin. The first temperature threshold may refer to a maximum temperature that the radiation device 541 can normally operate. As another example, if the temperature in the medical cabin is lower than a second temperature threshold, the temperature adjustment device 542 may raise the temperature in the medical cabin. The second temperature threshold may refer to a minimum temperature that the radiation device 541 can normally operate. The temperature adjustment device 542 may control the temperature in the medical cabin in a proper temperature range (e.g., from the second temperature threshold to the first temperature threshold). As still another example, if the humidity in the medical cabin is higher than a first humidity threshold, a dehumidifier may be turned on to lower the humidity in the medical cabin. The first humidity threshold may refer to a maximum humidity that the radiation device 541 can normally operate. As still another example, if the humidity in the medical cabin is lower than a second humidity threshold, a humidifier may be turned on to raise the humidity in the medical cabin. The second humidity threshold may refer to a minimum humidity that the radiation device 541 can normally operate. The humidity adjustment device 545 may control the humidity in the medical cabin in a proper humidity range (e.g., from the second humidity threshold to the first humidity threshold).

In some embodiments, the control device 560 may provide the feedback based on the altitude where the medical cabin is located monitored by the altitude detector 533 and the influence of different altitudes on the temperature adjustment device 542 and/or the humidity adjustment device 545 obtained from the storage device 570. The influence of different altitudes on the temperature adjustment device 542 and/or the humidity adjustment device 545 may indicate that an output power of one of the temperature adjustment device 542 and/or the humidity adjustment device 545 may be reduced with an increase in the altitude. For instance, a corresponding relationship between output powers of each of the temperature adjustment device 542 and the humidity adjustment device 545 and a plurality of altitudes may be stored in a storage. The control device 560 may retrieve an output power of one of the temperature adjustment device 542 and/or the humidity adjustment device 545 corresponding to an altitude based on the corresponding relationship. For example, the control device 560 may generate a prompt instruction to remind a user to adjust the temperature adjustment device 542 and/or the humidity adjustment device 545 based on the altitude where the medical cabin is located monitored by the altitude detector 533 and the influence of different altitudes on the temperature adjustment device 542 and the humidity adjustment device 545 obtained from the storage device 570. As another example, the control device 560 may generate an adjustment instruction for adjusting the temperature adjustment device 542 and/or the humidity adjustment device 545 based on the altitude where the medical cabin is located monitored by the altitude detector 533 and the influence of different altitudes on the temperature adjustment device 542 and the humidity adjustment device 545 obtained from the storage device 570.

In some embodiments, the radiation device 541 may be powered by the energy storage component 543. The energy storage component 543 may be powered by the power generation component 544. In some embodiments, the control device 560 may provide the feedback based on the altitude where the medical cabin is located monitored by the altitude detector 533, the influence of different altitudes on the power generation component 544 and/or the energy storage component 543, and the remaining volume of the fuel obtained from the storage device 570. The influence of different altitudes on the power generation component 544 and/or the energy storage component 543 may indicate that an output power of one of the power generation component 544 and the energy storage component 543 may be reduced with an increase in the altitude. For instance, a corresponding relationship between output powers of one of the power generation component 544 and the energy storage component 543 and a plurality of altitudes may be stored in a storage. The control device 560 may retrieve an output power of one of the power generation component 544 and the energy storage component 543 corresponding to an altitude based on the corresponding relationship. For example, at an altitude of 1000 meters, an output power of the power generation component 544 and/or the energy storage component 543 may be 60 KVA. At an altitude of 3000 meters, the output power of the power generation component 544 and/or the energy storage component 543 may be 50KVA. Therefore, when the power generation component 544 and/or the energy storage component 543 supplies power to the radiation device 541 at high altitudes, the endurance time and an exposure power of the radiation device 541 may be paid attention to. When the endurance time is lower than a time threshold, or an output power of the power generation component 544 or the energy storage component 543 is not enough to support the exposure at a current altitude, the control device 560 may provide feedback, such as a prompt instruction, an adjustment instruction for turning off the power generation component 544 or the energy storage component 543.

In some embodiments, the control device 560 may obtain the vibration parameters of the medical cabin from the vibration detector 534 or the storage device 570. The control device 560 may provide the feedback including a prompt instruction and/or an adjustment instruction associated with a damping apparatus 556, or one or more operation parameters (e.g., a speed) of the movable platform 510. The damping apparatus may include an air suspension 5561, a damper 5562, etc. For example, the vibration detector 534 disposed outside the medical cabin may detect vibrations of the medical cabin. The control device 560 may obtain the vibrations (e.g., a vibration intensity, a vibration acceleration) outside the medical cabin from the vibration detector 534 disposed outside the medical cabin or the storage device 570. The control device 560 may generate an adjustment instruction for controlling the damping apparatus (e.g., a damping coefficient of the air suspension 5561, a damping coefficient of the damper 5562) to adjust the vibration or one or more operation parameters of the movable platform 310. For example, the damping apparatus 556 may adjust operation parameters, e.g., a damping coefficient of the air suspension 5561, a damping coefficient of the damper 5562 to adjust the vibration of the medical cabin. In some embodiments, the damping apparatus may be disposed on an axle of the movable platform. The control device 560 may generate an adjustment command for automatically adjusting the damping coefficient of the damping apparatus. Therefore, the intensity of the vibration transmitted to the radiation device 541 may be reduced, avoiding damage of the radiation device 541 due to excessive vibrations.

In some embodiments, one or more second imaging devices disposed outside the medical cabin may be configured to acquire the monitoring result including the image data of the scene outside the medical cabin. The control device 560 may provide the feedback based on the monitoring result including the image data of the scene outside the medical cabin. For example, if the control device 560 determines that an external driving environment is complicated based on an image analysis technique and excessive vibrations will be caused, the control device 560 may generate a prompt instruction to remind a driver of the movable platform of the movable medical device that a current road is not suitable for the movable medical device, and a driving route should be changed in time.

In some embodiments, one or more first imaging devices disposed inside the medical cabin may be configured to acquire the monitoring result including the image data of the scene inside the medical cabin. The image data of the scene may include image data of the radiation device 541, image data of the couch, image data of the subject, etc. The control device 560 may provide the feedback based on the monitoring result including the image data of the scene outside the medical cabin. For example, the control device 560 may determine whether an anomaly exists in the scene inside the medical cabin. The anomaly may include a failure of the radiation device 541, the subject not being scanned correctly, etc. When the volume of fuel in the power generation component 544 is too low, the control device 560 may determine whether the scanning is stopped or power is stopped to supply to the radiation device 541 based on the monitoring result acquired by the first imaging device, such as whether there is a subject on the couch, whether the radiation device 541 is operating, etc. Therefore, the safety of the subject and/or the radiation device 541, the success rate of the scanning, and the quality of the image may be ensured.

In some embodiments, the control device 560 may determine whether the radiation device 541 includes an anomaly based on the operation state acquired by the state monitoring device (e.g., a malfunction of a tube (e.g., a filament of the tube, an anode target, a high-voltage generator), a malfunction of a collimator, a malfunction of a detector, etc.) and the data stored in the storage device 570. Further, the control device 560 may determine a reason for the anomaly in response to determining that the radiation device 541 includes the anomaly.

In some embodiments, the feedback receiving components 540 may include an air purifier or an air cleaner (not shown in FIG. 5). The air purifier may be configured to remove contaminants from the air in the medical cabin to improve the air quality of the medical cabin.

In some embodiments, the monitoring system 500 may further include a storage device 570. The monitoring result acquired by the one or more monitoring devices 530, the feedback provided by the control device 560, etc., may be stored in the storage device 570. In some embodiments, the processing device 120 may determine, based on data stored in the storage device 570, a reason of an anomaly in response to determining that the radiation device 541 includes the anomaly. For example, if the movable medical device is moved to an area with a high altitude, or the temperature and humidity do not meet predetermined requirements, the processing device 120 may determine, based on data stored in the storage device 570, the anomaly is caused by a system faulty or an external factor. In some embodiments, the storage device 570 may include historical operation data. The processing device 120 may determine an anomaly by comparing operation parameters at a current time and historical operation parameters in the historical operation data.

In some embodiments, the monitoring system 500may further include a communication apparatus 580. The communication apparatus 580 may be configured to transmit the monitoring result and the feedback to a remote device. Therefore, the monitoring result acquired by the one or more monitoring devices and the feedback provided by the control device 560 may be shared with the remote device. The remote device may include a server, other terminal devices, etc., which are communicatively connected with the control device 560. The communication apparatus 580 may be a portion of the control device 560 or be disposed of independently of the control device 560. The communication apparatus 580 may be a wired communication apparatus or a wireless communication apparatus. The wired communication apparatus may include communication ports, such as a universal serial bus (USB), a controller area network (CAN), serial and/or other standard network connections, an inter-integrated circuit (I2C) bus, etc. The wireless communication apparatus may use any type of wireless communication system, for example, Bluetooth, infrared, wireless fidelity (WiFi), cellular technology, satellite, broadcasting, etc. The cellular technology may include mobile communication technologies such as a second generation (2G), a third generation (3G), a fourth generation (4G), a fifth generation (5G), etc.

In some embodiments, the monitoring system 500 may further include a positioning device 590. The positioning device 590 may be configured to determine a current location (e.g., longitude information, latitude information, altitude information) of the movable medical device. The control device 560 may determine a recommended route based on the current location, a destination location, and environment parameters of multiple routes stored in the storage device 570 or the remote device. The control device 560 may provide feedback to prompt the recommended route. The positioning device 590 may be a positioning device, such as a global positioning system (GPS). For example, there are multiple routes from the current location to the destination location predetermined by the movable medical device. Environment parameters of multiple routes may be stored in the storage device 570 or the remote device. The control device 560 may determine that there are multiple optional routes based on the movable medical device and the destination location. Accordingly, the control device 560 may determine a recommended route that is most suitable for the movable medical device according to the environmental parameters of each optional route, such as relatively flat road conditions, fewer obstacles, etc. The control device 560 may provide a prompt instruction for prompting the recommended route to prompt the driver to select the recommended route.

It should be noted that the description of the monitoring device 500 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments.

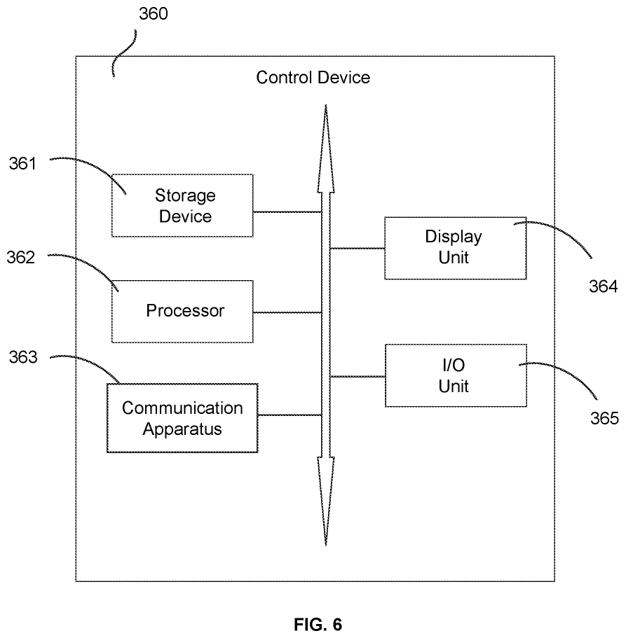

FIG. 6 is a schematic diagram illustrating an exemplary structure of a control device 360 according to some embodiments of the present disclosure.

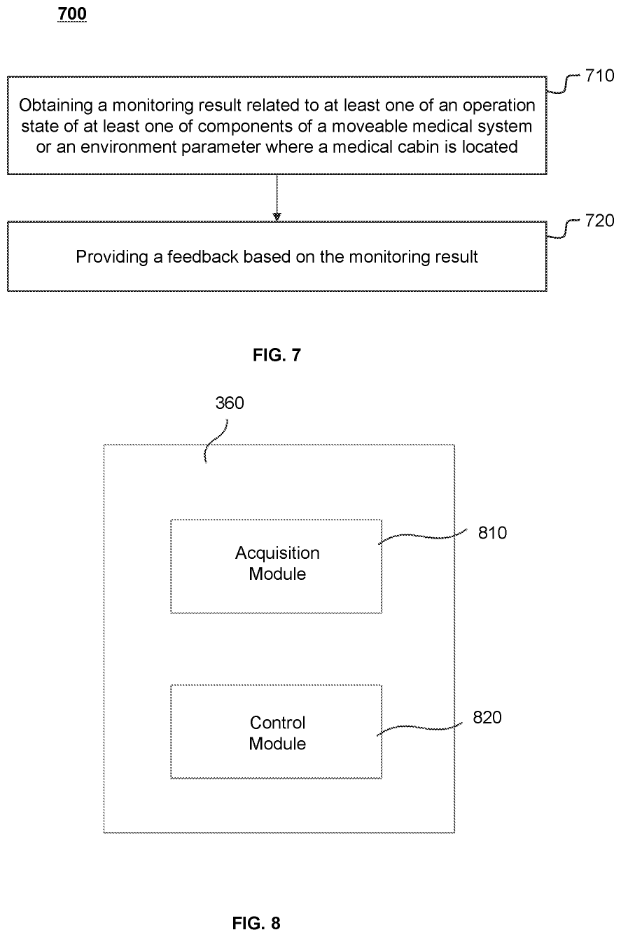

The control device 360 in the embodiment may refer to a device that can automatically perform numerical calculation and/or information processing according to predetermined or stored instructions. The control device 360 may include a storage device 361, a processor 362, and a computer program stored in the storage device 361 and operated in the processor 362. The processor 362 may implement the process of monitoring a movable medical device according to some embodiments of the present disclosure when the computer program is executed (as shown in FIG. 7).

The control device 360 may be any electronic product that can perform data processing, such as a personal computer (PC), a tablet computer, a smartphone, a personal digital assistant (PDA), or the like, or any combination thereof.

The storage device 361 may be configured to store computer programs and/or modules. The processor 362 may implement various functions of the control device 360 by operating or executing the computer programs and/or modules stored in the storage device 361 and retrieving data stored in the storage device 361. The storage device 361 may include a program storage area and a data storage area. The program storage area may store an operating system, an application program required by at least one function (e.g., a sound playback function, an image playback function, etc.), etc. The data storage area may store data (e.g., audio data, telephone book, etc.) created by the use of the control device 360. In addition, the storage device 361 may include a high-speed random access memory, a non-volatile memory, etc., such as a hard disk, a memory, a plug-in hard disk, a smart media card (SMC), a secure digital (SD) card, a flashcard, at least one magnetic disk memory, a flash memory, or other volatile solid-state storage devices.

The processor 362 may include a central processing unit (CPU), a general-purpose processor, a digital signal processor (DSP), an application-specific integrated circuit (ASIC), a field-programmable gate array (FPGA), a programmable logic device, a discrete gate, a transistor logic device, a discrete hardware component, etc. The general-purpose processor may include a microprocessor or any conventional processor. The processor 362 may be a control center of the control device 360. The processor 362 may be connected with each portion of the control device 360 through various interfaces and lines.

In some embodiments, the processor 362 may retrieve program codes stored in the storage device 361 to perform related functions. For example, each module in FIG. 5 may be a program code stored in the storage device 361 and executed by the processor 362 to implement a control method of the present disclosure (e.g., the method for monitoring the movable medical device shown in FIG. 5 in the embodiment).

The control device 360 may further include at least one communication unit 363, at least one display unit 364, at least one input/output (I/O) unit 365, and at least one communication bus. The communication bus may be configured to realize the connection and communication between these components. The communication unit 363 may be configured to cause the control device 360 to communicate with other devices or to obtain designated information on the network, for example, to communicate with a management system set up in the hospital. The display unit 353 may be configured to display prompt information associated with one or more monitoring results of the monitoring system. The I/O unit 365 may be configured to allow a user (e.g., a server provider, a doctor, an operator, etc.) to interact with the control device 360 by inputting control instructions, such as turning on/off a device, adjust operation states of the device, adjust a parameter of the device, etc.

The communication unit 363 may include a wired communication device, a wireless communication device, or a combination thereof. The wired communication device may include a communication port, such as a universal serial bus (USB), a controller area network (CAN), serial and/or other standard network connections, an inter-integrated circuit (I2C) bus, etc. The wireless communication device may include any type of wireless communication systems, such as Bluetooth, infrared, wireless fidelity (WiFi), cellular technology, a satellite, broadcasting, etc. The cellular technology may include mobile communication technologies such as a second generation (2G), a third generation (3G), a fourth generation (4G), a fifth generation (5G), etc.

The display unit 364 may include a liquid crystal display (LCD), a light emitting diode (LED) display, an organic light-emitting diode (OLED), or the like.

The I/O unit 365 may include any suitable input device and any suitable output device. Exemplary input devices may include a mouse, a keyboard, a touch screen, a non-contact input (e.g., a gesture input, a voice input, etc.), etc. Exemplary output devices may include a speaker, etc.

It should be noted that, for those skilled in the art, the schematic diagram is merely an example of the control device 360 and not intended to limit the scope of the present disclosure. For example, the control device 360 may also include an input/output unit, a network access unit, or the like.

In some embodiments, the computer program stored by the control device 360 may be divided into one or more modules/units. The one or more modules/units may be stored in the storage device 361 and executed by the processor 362 according to the present disclosure. One or more modules/units may be a series of computer program instructions capable of completing specific functions. The instructions may be used to describe the execution process of the computer program in the control device 360.

FIG. 7 is a flowchart illustrating an exemplary process for monitoring a movable medical device according to some embodiments of the present disclosure. In some embodiments, process 700 may be executed by the movable medical system (e.g., the movable medical system 100). For example, the process 700 may be implemented as a set of instructions (e.g., an application) stored in a storage device (e.g., the storage device 130, the storage device 361, and/or the storage device 570). In some embodiments, the processing device 120, or the control device 360 (e.g., the processor 351 illustrated in FIG. 6) may execute the set of instructions and may accordingly be directed to perform the process 700. The operations of the illustrated process presented below are intended to be illustrative. In some embodiments, the process 700 may be accomplished with one or more additional operations not described and/or without one or more of the operations discussed. Additionally, the order of the operations of process 700 illustrated in FIG. 7 and described below is not intended to be limiting.

In 710, the control device 360 (e.g., the acquisition module 810) may obtain a monitoring result related to at least one of an operation state of at least one of components of a movable medical device or an environment parameter where a medical cabin is located.

The operation state may include a normal operation, an anomaly, etc., of at least one of components of the movable medical device. Exemplary anomalies may include that operation parameters do not satisfy one or more conditions, a malfunction of a portion of a device (e.g., a tube (e.g., a filament of the tube, an anode target, a high-voltage generator), a collimator, a detector, etc.), etc. The operation state may be determined based on operation parameters and/or image data. The operation parameter may include remain energy of an energy storage component of the movable medical device, a current volume of fuel in a fuel tank of a power generation component of the movable medical device, or the like, or any combination thereof. The image data may include image data of a medical device, image data of a scene inside/outside the medical cabin, or the like, or any combination thereof. The environment parameter may include a temperature inside/outside the medical cabin, a humidity inside/outside the medical cabin, an altitude where the medical cabin is located, a vibration amplitude of the medical cabin, a vibration frequency of the medical cabin, a speed of the medical cabin, air quality indexes, or the like, or any combination thereof. The monitoring result may be acquired by one or more monitoring devices. The one or more monitoring devices may include a temperature detector, a humidity detector, an altitude detector, a vibration detector, a speed detector, an imaging device, an energy detector, a state monitoring device, or the like, or any combination thereof. More descriptions regarding the one or more monitoring devices and the acquisition of the monitoring result may be found elsewhere in the present disclosure (e.g., FIG. 5 and the descriptions thereof).

In some embodiments, the monitoring result may include data associated with an operation parameter of a component (e.g., of the movable medical device and/or data associated with the environment parameter. For example, the monitoring result may include a temperature inside/outside the medical cabin, a humidity inside/outside the medical cabin, an altitude where the medical cabin is located, the vibration amplitude of the medical cabin, a vibration frequency of the medical cabin, a speed of the medical cabin, air quality indexes, remaining energy of an energy storage component of the movable medical device, a current volume of fuel in a fuel tank of a power generation component of the movable medical device, image data of a medical device, image data of a scene inside/outside the medical cabin, or the like, or any combination thereof.

In some embodiments, the monitoring result may indicate whether the operation parameter of a component and/or an environment parameter satisfy a condition.

In some embodiments, the control device 360 may obtain the monitoring result from the one or more monitoring devices. For example, after the one or more monitoring devices acquire the monitoring result, the control device 360 may obtain the monitoring result from the one or more monitoring devices directly. In some embodiments, the control device 360 may obtain the monitoring result from a storage device (e.g., the storage device 130, the storage device 361, the storage device 370, and/or a remote device). For example, after the one or more monitoring devices acquires the monitoring result, the one or more monitoring devices may transmit the monitoring result to a storage device (e.g., the storage device 130, the storage device 361, the storage device 370, and/or a remote device). The control device 360 may retrieve the monitoring result from the storage device.

In 720, the control device 360 may provide feedback based on the monitoring result. In some embodiments, the feedback may include a prompt instruction associated with the monitoring result, an adjustment instruction for adjusting an operation of one or more components related to the monitoring result, or the like, or any combination thereof.

The prompt instruction may include a situation of a portion of the movable medical device, a reminder for a user, a recommendation to advise the user. The prompt instruction may be used to transmit to a displayer (e.g., the display unit 364) for display. Alternatively, the prompt instruction may be used to remind the user to perform the prompt instruction.

The adjustment instruction for adjusting an operation of one or more components related to the monitoring result may include causing the one or more components to adjust based on the adjustment instruction. For example, the adjustment instruction may be transmitted to a component (e.g., the feedback receiving devices 540), the component may be caused to adjust based on the adjustment instruction. The one or more components receiving the adjustment instruction and the prompt instruction may also be referred to as feedback receiving devices. For example, after the control device 360 obtains the monitoring result, the control device 360 may determine the feedback by determining whether the monitoring result satisfies one or more conditions. The one or more conditions may be that a remaining volume of fuel is lower than a fuel threshold, the temperature in the medical cabin is lower or higher than a temperature threshold, the humidity in the medical cabin is lower or higher than a humidity threshold, a vibration acceleration reaches a vibration threshold, etc. More descriptions regarding providing the feedback may be found elsewhere in the present disclosure, for example, FIG. 5 and relevant descriptions thereof.

In some embodiments, the control device 560 may be configured to cause the feedback receiving devices to adjust the operation state of the feedback receiving devices and/or the environment parameter where the medical cabin is located according to the feedback. For example, the control device 560 may determine whether the remaining volume of the fuel in the fuel tank is lower than the volume threshold based on the current volume of the fuel in the fuel tank of the power generation component monitored by the energy detector. If the current volume of the fuel is lower than the volume threshold, the control device 560 may generate and output the feedback including a prompt instruction indicating prompt information related to the fuel shortage, a prompt instruction to turn off the power generation component, etc. As another example, the control device 560 may provide the feedback based on the altitude where the medical cabin is located monitored by an altitude detector and an influence of different altitudes on the feedback receiving devices. The influence of different altitudes on the feedback receiving devices may indicate that an output power of one of feedback receiving devices may be reduced with an increase in the altitude. The control device 560 may increase the output power of one of feedback receiving devices with the increase of the altitude. As still another example, the control device 560 may provide the feedback to adjust the temperature adjustment device and/or the humidity adjustment device based on the temperature and/or humidity inside/outside the medical cabin from the temperature detector and/or the humidity detector or the storage device 570. More descriptions regarding the adjustment of the feedback receiving devices may be found elsewhere in the present disclosure, for example, FIG. 5 and relevant descriptions thereof.

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the operation 720 may be divided into two or more sub-operations.

FIG. 8 is a block diagram illustrating an exemplary control device 360 according to some embodiments of the present disclosure. The control device 360 may include an acquisition module 810 and a control module 820. The module in some embodiments of the present disclosure refers to a series of computer programs that can be executed by a computer and can perform functions.

The acquisition module 810 may be configured to acquire a monitoring result related to at least one of an operation state of at least one of components of a movable medical device or an environment parameter where a medical cabin is located. In some embodiments, the monitoring result may include data associated with an operation parameter of a component (e.g., of the movable medical device and/or data associated with the environment parameter. In some embodiments, the monitoring result may indicate whether the operation parameter of a component and/or an environment parameter satisfy a condition. More descriptions regarding the obtaining of the monitoring result may be found elsewhere in the present disclosure, for example, operation 710 in FIG. 7 and relevant descriptions thereof.

The control module 820 may be configured to provide feedback based on the monitoring result. In some embodiments, the feedback may include a prompt instruction associated with the monitoring result, an adjustment instruction for adjusting an operation of one or more components related to the monitoring result, or the like, or any combination thereof. The prompt instruction may include a situation of a portion of the movable medical device, a reminder for a user, a recommendation to advise the user. The adjustment instruction for adjusting an operation of one or more components related to the monitoring result may include causing the one or more components to adjust based on the adjustment instruction. More descriptions regarding the providing the feedback may be found elsewhere in the present disclosure, for example, operation 720 in FIG. 7 and relevant descriptions thereof.

It should be noted that the above description of the control device 360 is merely provided for the purposes of illustration, and not intended to limit the scope of the present disclosure. For persons having ordinary skills in the art, multiple variations or modifications may be made under the teachings of the present disclosure. However, those variations and modifications do not depart from the scope of the present disclosure. For example, the control module 820 may be divided into a plurality of sub-modules. Each of the plurality of sub-modules may perform a portion of the operation 720.

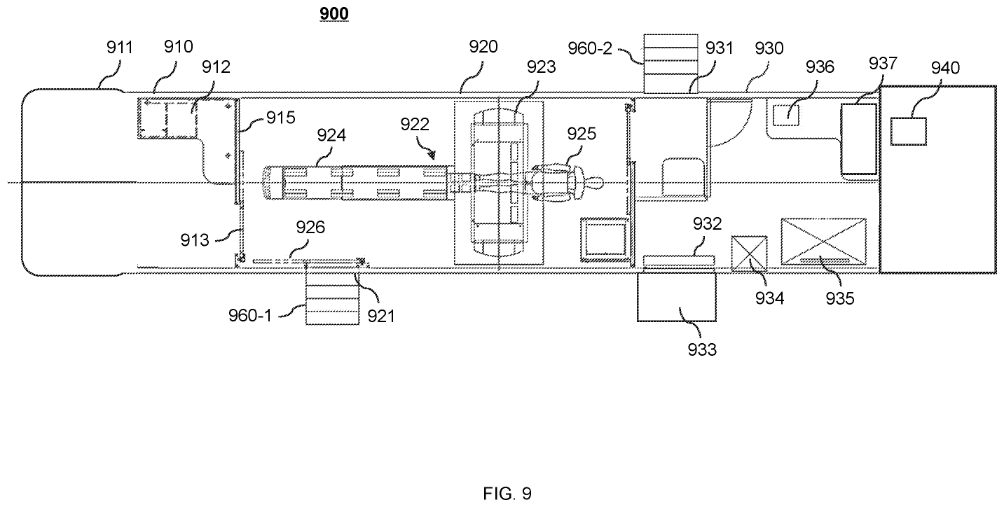

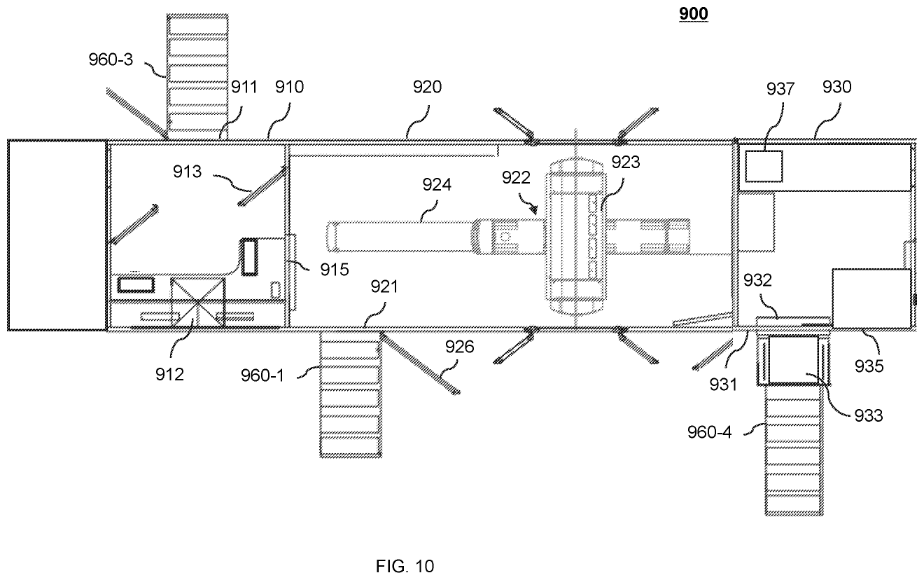

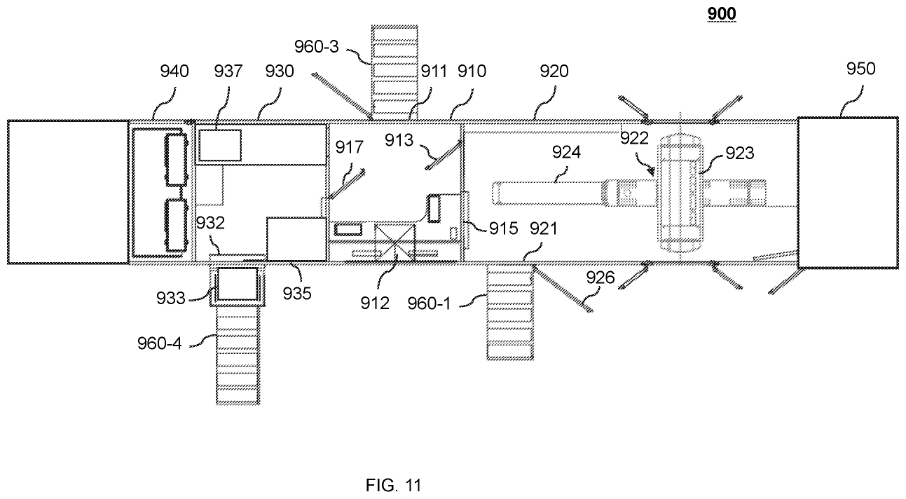

FIG. 9 is a schematic diagram illustrating an exemplary movable medical device according to some embodiments of the present disclosure. FIG. 10 is a schematic diagram illustrating another exemplary movable medical device according to some embodiments of the present disclosure. FIG. 11 is a schematic diagram illustrating still another exemplary movable medical device according to some embodiments of the present disclosure. In some embodiments, a movable medical device may be an example of the movable medical device as described in FIG. 1, the movable medical device as described in FIG. 2, or the movable medical device as described in FIG. 3. The movable medical device 900 may be described below with reference to FIGS. 9-11. It should be noted that the descriptions of the movable medical device are merely provided for illustration, and not intended to limit the scope of the present disclosure.

As shown in FIGS. 9-11, the movable medical device 900 may include a movable platform and a medical cabin.

The movable platform may be configured to move the medical cabin to a desired location. Exemplary movable platforms may include a van, a coach, a truck, a multi-purpose vehicle, a ship, an aircraft, etc. For example, the movable platforms as shown in FIGS. 9 and 10 may be a vehicle. As another example, the movable platform as shown in FIG. 11 may be a truck. More descriptions regarding the movable platform may be found elsewhere in the present disclosure (e.g., FIG. 3 and the descriptions thereof).

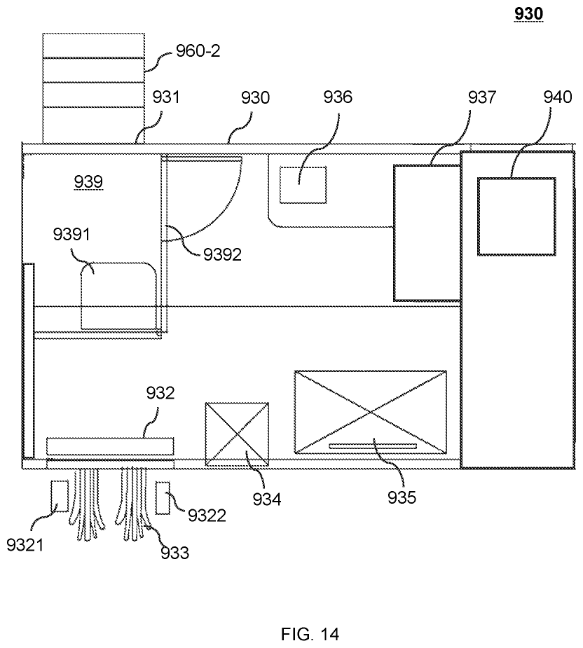

The medical cabin may be configured to accommodate medical resources for providing multiple types of medical services. In some embodiments, the medical cabin may include a radiation room 920, a sample processing room 930, and a control room 910. The radiation room 920 may be configured to accommodate a radiation device 922 for providing medical imaging and/or treating service. The radiation device 922 may be a medical device, for example, a positron emission tomography (PET) device, a single-photon emission computed tomography (SPECT) device, a computed tomography (CT) device, a magnetic resonance imaging (MRI) device, a radiation therapy (RT) device, or the like, or any combination thereof. The sample processing room 930 may be configured to accommodate medical resources for providing a sampling service, a sample detection service, or the like, or any combination thereof. More descriptions regarding the sample processing room 930 may be found elsewhere in the present disclosure (e.g., FIGS. 14-16 and the descriptions thereof). The control room 910 may be configured to accommodate a control platform 912 used to control the radiation device 922 for providing the medical imaging service. In some embodiments, the control room 910 and the radiation room 920 may be located adjacent to each other. For example, the control room 910 may be located between the radiation room 920 and the sample processing room 930. As another example, the radiation room 920 may be located between the control room 910 and the sample processing room 930.

In some embodiments, the medical cabin may be disposed on the movable platform. Therefore, the medical cabin may be moved, by the movable platform, to the desired location. For example, the medical cabin may be a shelter structure (e.g., a container-like structure) that can be transported and placed as a whole by the movable platform. As another example, the medical cabin may be assembled from multiple portions. The radiation room 920, the sample processing room 930, and the control room 910 may be three portions independent of each other. The three portions may be separately transmitted to the desired portion and assembled to form the medical cabin. In some embodiments, the medical cabin may be built (e.g., assembled) on site. For example, after a desired location is determined, a medical cabin (e.g., a radiation device and devices for sample processing) may be integrated into a movable platform to form the movable medical device 900. In some embodiments, the medical cabin may be fixed on the movable platform. That is, the medical cabin and the movable platform may be regarded as a whole. Therefore, the movable medical device 900 may be moved to the desired location in time, which may easily and quickly complete an equipment deployment, and solve emergency needs (e.g., a hospital, a community, a school, a factory, etc.).

In some embodiments, the control room 910 may include a first door 911. The radiation room 920 may include a second door 921. The first door 911 may be configured to allow one or more service providers (e.g., a doctor, an operator, etc.) to enter and exit the control room 910. The second door 921 may be configured to allow a service receiver (e.g., a subject to be scanned, a patient, etc.) to enter and exit the radiation room 920. By setting the first door 911 and the second door 921 for the control room 910 and the radiation room 920, respectively, contact between the service provider and the service receiver may be reduced, thereby avoiding cross-infection of pathogens.

In some embodiments, a first distance (e.g., a straight-line distance, a moving distance) between the first door 911 and the second door 921 may be greater than a threshold (also referred to as a distance threshold). The distance threshold may be 2 meters, 2.5 meters, 3 meters, 5 meters, etc. In some embodiments, a size of the distance threshold may be determined based on a type of pathogens that is detected. For example, a distance threshold of a movable medical device 900 for detecting the novel coronavirus may be greater than a distance threshold of a movable medical device for detecting the influenza A virus. The first distance between the first door 911 and the second door 921 may cause contact between the service provider and the service receiver and possible cross-infection may be avoided effectively.

In some embodiments, an isolation device (also referred to as a first isolation device) may be provided in a space between the first door 911 and the second door 921. The isolation device may be configured to prevent personnel (e.g., the service provider, the service receiver, etc.) or any other undesired object (e.g., pathogens) to pass through the space. In some embodiments, the first isolation device may include a physical isolation component disposed between the first door 911 and the second door 921. For example, the first isolation device may include a foldable screen. The foldable screen may be foldably disposed on an outside of the medical cabin. When the foldable screen is unfolded, the first door 911 and the second door 921 may be blocked by the foldable screen. In some embodiments, the first isolation device may include a disinfection isolation component. The disinfection isolation component may be configured to disinfect pathogens in a certain area (e.g., the space between the first door 911 and the second door 921. For example, the disinfection isolation component may include a disinfectant spraying device, an ultraviolet lamp, or the like. The first isolation device may be set to effectively avoid the possible cross-infection between the service provider who performs or provides the medical service and the service receiver who receives the medical service. In some embodiments, the first distance between the first door 911 and the second door 921 may be greater than the first distance threshold, and the isolation device may be provided in the space between the first door 911 and the second door 921. The space between the first door 911 and the second door 921 may indicate a space from the second door 921 and the first door 911 around the movable medical device 900.

In some embodiments, the first door 911 and the second door 921 may be located at different sides of the medical cabin. For example, the first door 911 and the second door 921 may be arranged at adjacent sides of the medical cabin. As another example, in the embodiments shown in FIGS. 9-11, the first door 911 and the second door 921 may be disposed at opposite sides of the medical cabin. The first door 911 and the second door 921 may be disposed at different sides of the medical cabin, which may effectively prevent the contact and the possible cross-infection between the service provider entering and exiting from the first door 911 and the service receiver entering and exiting from the second door 921. In addition, the service provider and the service receiver may be diverted and managed during medical operations.

In some alternative embodiments, the control room 910 and the radiation room 920 may be entered through the same door. For example, a service provider may enter the control room 910 through the second door 921 of the radiation room 920 and a passage (e.g., a first channel 916 as shown in FIG. 9) between the radiation room 920 and the control room 910. As another example, a service receiver may enter the radiation room 920 through the first door 911 of the control room 910 and a passage (e.g., the first channel 916 as shown in FIG. 9) between the radiation room 920 and the control room 910. In this case, the medical cabin may be used to provide the medical imaging service to a non-infectious service receiver. Alternatively, the service provider may protect against pathogens through a protective measure (e.g., wearing protective clothing, disposing an isolation room, etc.).

In some embodiments, the sample processing room 930 may include a third door 931. The third door 931 may be used for the service provider (e.g., medical staff, a service provider) to enter and exit the sample processing room 930. In some embodiments, the second door 921 and the third door 931 may be located at different sides of the medical cabin. For example, the second door 921 and the third door 931 may be disposed on adjacent sides of the medical cabin. As another example, the second door 921 and the third door 931 may be disposed on opposite sides of the medical cabin. As shown in FIG. 9, the first door 911 and the third door 931 may be located at the same side of the medical cabin, and the second door 921 may be located at an opposite side of the side where the first door 911 and the third door 931. The third door 931 and the second door 921 may be located at different sides of the medical cabin, which may effectively prevent the contact and the possible cross-infection between the service provider entering and exiting from the third door 931 and the service receiver entering and exiting from the second door 921. In addition, the service provider and the service receiver may be diverted and managed during medical operations. In some embodiments, the sample processing room 930 may not be provided with a door directly leading to the outside of the medical cabin. For example, in the embodiment shown in FIG. 11, the sample processing room 930 and the control room 910 may be arranged adjacently. An access door 917 may be located in an isolation wall between the sample processing room 930 and the control room 910. The access door may be connected to the sample processing room 930 and the control room 910. Therefore, the service provider may enter the sample processing room 930 through the access door 917, the first door 911, and the control room 910.

In some embodiments, a second distance (e.g., a straight-line distance, a moving distance) between the first door 911 and the third door 931 may be greater than the distance threshold. A third distance (e.g., a straight-line distance, a moving distance) between the second door 921 and the third door 931 may be greater than the distance threshold. In some embodiments, the first distance, the second distance, and the third distance may be the same or different. For example, the first distance, the second distance, and the third distance may be further determined based on a count of service receivers at each door, a size of the movable medical device 900, a shape of the movable medical device 900, etc.

In some embodiments, an isolation device (also referred to as a second isolation device) may be provided in a space between the first door 911 and the third door 931. An isolation device (also referred to as a third isolation device) may be provided in a space between the second door 921 and the third door 931. The second isolation device may be configured to prevent personnel (e.g., the service provider, the service receiver, etc.) to pass through the space between the first door 911 and the third door 931. The third isolation device may be configured to prevent personnel (e.g., the service provider, the service receiver, etc.) to pass through the space between the second door 921 and the third door 931. In some embodiments, types of the three isolation devices may be the same or different. For example, a type of each of the three isolation devices may be further determined based on a count of service receivers at each door, a size of the movable medical device 900, a shape of the movable medical device 900, a terrain where the movable medical device 900 is located, etc.

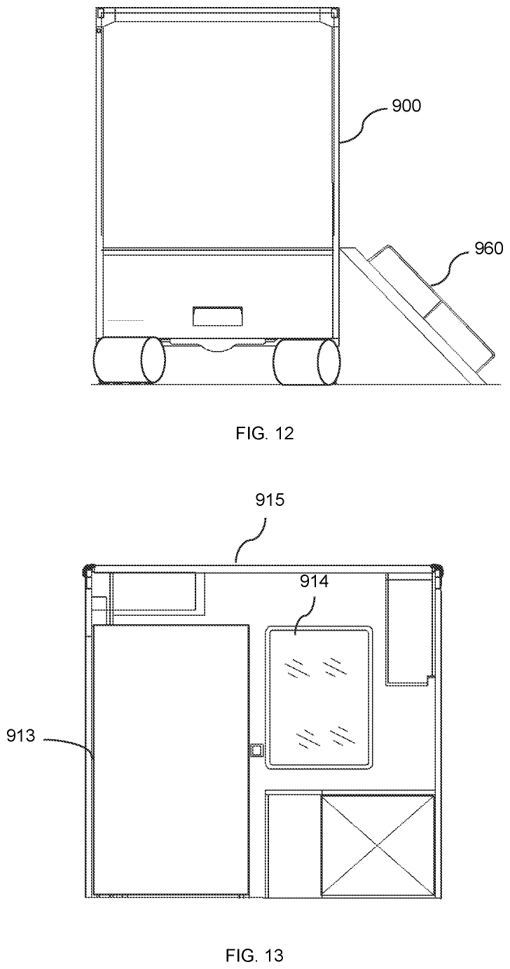

In some embodiments, when a door (e.g., the first door 911, the second door 921, and the third door 931) of the medical cabin is at a distance from the ground, a stair 960 may be disposed at the door of the medical cabin. Referring to FIG. 12, FIG. 12 is a schematic diagram illustrating a rear view of a medical cabin according to some embodiments of the present disclosure. As shown in FIG. 12, in some embodiments, the stair 960 may be a component independent of the medical cabin. The stair 960 may be connected with a bottom portion of the outside of the door of the medical cabin. In some embodiments, the stair 960 may be connected with the outside of the medical cabin in a foldable manner. The stair 960 may be unfolded when needed. Alternatively, the stair 960 may be folded and retracted when not needed. In the embodiment shown in FIG. 9, a stair 960-1 may be disposed at the second door 921, and a stair 960-2 may be disposed at the third door 931. In the embodiments shown in FIGS. 10-11, a stair 960-3 may be disposed at the first door 911, and a stair 960-1 may be disposed at the second door 921. In some embodiments, as shown in FIGS. 10-11, a stair 960-4 may also be located in front of a sampling window 932, so that the service receiver to be detected may be close to the sampling window 932.

In some embodiments, an identity recognition device may be located outside the medical cabin. In some embodiments, the identity recognition device may be configured to identify a service provider (e.g., a doctor, a nurse, an operator, etc.). In some embodiments, the identity recognition device may be located at a door of the medical cabin. For example, each of the first door 911, the second door 921, and/or the third door 931 may be located with an identity recognition device. A person (e.g., the operator, the service receiver, the medical staff, etc.) that has been identified by the identity recognition device may enter through the door of the medical cabin corresponding to the identity recognition device. In some embodiments, the identity recognition device may be connected with a gate corresponding to the door. When a person is identified by the identity recognition device, the gate corresponding to the door may be opened/unlocked. The identity recognition device may be disposed to effectively manage the entering and existence of the personnel, which may reduce the contact between the service provider (e.g., the operator, the medical staff, etc.) and the service receiver. In addition, the service receiver may be prevented from entering by mistake (e.g., without reading a sign at the door, no sign at the door, etc.). For example, a service receiver to be scanned may be prevented from entering the control room 910 by mistake.

In some embodiments, the identity recognition device may be used to recognize the identity of the service provider in a contactless recognition. For example, the identity recognition device may include a face recognition device, an iris recognition device, a palmprint recognition device, a voiceprint recognition device, a voice recognition device, or the like, or any combination thereof. In some embodiments, reserved verification information of the service provider may be stored in the identity recognition device. For example, the reserved verification information may include facial information, iris information, palmprint information, voiceprint information, voice information, or the like, or any combination thereof., of the service provider. When user information of the service provider recognized by the identity recognition device is consistent with the reserved verification information, the service provider may be verified and permitted to enter the corresponding door. In some embodiments, the identity recognition device may send the recognized user information to a remote server for verification. The identity recognition device may determine whether the identity of the service provider is verified according to a received verification result. By disposing an identity recognition device for contactless recognition, the direct or indirect contact between the service provider and the service receiver may be effectively avoided, thereby preventing the cross-infection.

In some alternative embodiments, the identity recognition device may also include a fingerprint recognition device, a magnetic card recognition device, an integrated circuit (IC) card recognition device, a barcode recognition device, a quick response (QR) code recognition device, etc. The service provider may be recognized by using a work card, a social security card, an ID (identity) card, a medical consultation card, a paper or mobile phone barcode, a paper or mobile phone QR code, etc. In some embodiments, a single identity recognition device (e.g., a face recognition device) may be disposed to perform the identity recognition. In some embodiments, an identity recognition device that integrates multiple recognition manners may also be disposed to perform identity recognition. For example, an identity recognition device may be used to perform face recognition, fingerprint recognition, ID card recognition, etc., simultaneously.

In some embodiments, the identity recognition device may be further configured to recognize a service receiver (e.g., a subject to be scanned, a subject to be detected) based on information that the service receiver has reserved or registered. For example, when a service receiver is not within a reserved time, or an item (e.g., a nucleic acid detection) reserved by the service receiver does not match an item (e.g., a CT scanning) corresponding to the identity recognition device, the service receiver may not be verified by the identity recognition device. In this case, the identity recognition device may prompt the service receiver why the service receiver is not verified. In some embodiments, the identity recognition device may be further configured to recognize the service receiver based on information of the service receiver, such as information indicating whether the service receiver is routed or in an area including person infected by an infectious disease, whether or not the service receiver has been vaccinated, etc. Exemplary information may include quick response codes in different colors. For example, a quick response code in red may indicate that a service receiver has been contacted with the infected person or area. A quick response code in yellow may indicate that a service receiver is routed or in an area including a person infected by an infectious disease. A quick response code in green may indicate that a service receiver is with little risk of infection. Therefore, a service receiver includes a quick response code in red or a quick response code in yellow may be verified by the identity recognition device.

In some embodiments, the control platform 912 may be located in the control room 910. The radiation device 922 may be disposed in the radiation room 920. The control platform 912 may be used to control the radiation device 922 to scan the service receiver automatically or according to instructions of the service provider. In some embodiments, the control platform 912 may be implemented by a combination of hardware and software. The hardware portion of the control platform 912 may include a processor, a storage device, an input device, an output device, a communication device, or the like, or any combination thereof. The input device may include a mouse, a keyboard, a control button, a voice input device, an image input device, or the like, or any combination thereof. The output device may include a displayer, an audio output device, an indicator light, or the like, or any combination thereof. The software portion of the control platform 912 may be stored in a storage device and executed by an instruction execution system (e.g., a processor).

In some embodiments, as shown in FIGS. 9-12, the radiation device 922 may include a main body 923 and a couch 924. A service receiver 925 may lie on the couch 924. The service receiver 925 may be scanned by the radiation device 922. More descriptions regarding the service receiver may be found elsewhere in the present disclosure (e.g., FIG. 1 and the descriptions thereof). The service receiver 925 may be moved, through the couch 924, into the main body 923 to be scanned. In some embodiments, the radiation room 920 (or outside the radiation room 920) may further include a power supply system 950 (as shown in FIG. 11). The power supply system 950 may be configured to provide power for components (e.g., the radiation device 922) in the movable medical device 900. For example, the power supply system 950 may be used to supply power to the radiation device 922 to ensure the operation of the radiation device 922. The power supply system 950 may include a power distribution cabinet, a battery cabinet, a power generator, a control cabinet of a power generator, a power sleep controller (PSC), or the like, or any combination thereof. In some embodiments, the power supply system 950 may include a first electric source system, a second electric source system, and a control component. The first electric source system and the second electric source system may be configured to provide power to the radiation device 922. The control component may be configured to determine an operation state of each of the first electric source system and the second electric source system and control an operation of the each of the first electric source system and the second electric source system based on operation state. More descriptions regarding the power supply apparatus may be found elsewhere in the present disclosure (e.g., FIGS. 17-20 and the descriptions thereof).

An isolation wall 915 may be disposed in a space between the control room 910 and the radiation room 920. Referring to FIG. 13, FIG. 13 is a schematic diagram illustrating an isolation wall according to some embodiments of the present disclosure. As shown in FIG. 13, an observation window 914 may be disposed on the isolation wall 915 to facilitate the service provider in the control room 910 to observe a situation (e.g., position, posture, behavior, action, emotion, etc.) in the radiation room 920. In some embodiments, a connection door 913 may also be disposed on the isolation wall 915 to facilitate the service provider to enter and exit the radiation room 920. In some embodiments, walls of the radiation room 920 may be disposed with radiation shielding layers to shield the radiation in the radiation room 920. For example, six surfaces (i.e., a top surface, a bottom surface, and four side surfaces) of the radiation room 920 may be disposed with radiation shielding layers to shield the radiation in the radiation room 920. The radiation shielding layer may include a lead shielding layer. The observation window 914 may include a leaded glass. The doors (e.g., the connection door 913, the second door 921) on side surfaces (e.g., the isolation wall 915, the side surface including the second door 921, etc.) may include lead shielding layers.

In some embodiments, an electrical control gate 926 (e.g., an electric control slip gate) may be located at the second door 921. The electrical control gate 926 may be opened and closed by a control of the service provider (e.g., a voice control device, an optical control, password control, manual control, etc.). Alternatively, the electrical control gate 926 may be automatically opened and closed according to the recognition result of the identity recognition device. In some embodiments, a voice device may be disposed between the control room 910 and the radiation room 920, so that the service provider may be in contactless communication with the service receiver.

In some embodiments, one or more image acquisition devices may be in the radiation room 920. The image acquisition devices may be configured to acquire image data of the scene (e.g., the service receiver) in the radiation room 920. For example, the image acquisition device may acquire image data of a service receiver on the couch 924 and send the acquired image data to the control platform 912. The control platform 912 may be configured to control the radiation device 922 for providing the medical imaging service based on the image data. The image acquisition device may include an optical camera, an infrared camera, a depth camera, or the like, or any combination thereof. In some embodiments, the image acquisition device may be disposed on the sidewalls, the top wall, etc. of the radiation room 920.

In some embodiments, the control platform 912 may determine, based on the image data, a scanning region of the service receiver. The control platform 912 may further control, based on the scanning region, the medical imaging service to scan the service receiver. For example, the control platform 912 may control, based on the scanning region, the radiation device 922 to scan a service receiver on the couch 924. By setting the image acquisition device, the control platform 912 may determine, based on the image data, the position of the service receiver on the couch 924, and automatically determine the scanning region, which may effectively improve the efficiency of the medical scanning, reduce technical requirements of the service provider, and avoid the contact between the service provider and the service receiver.

In some embodiments, the scanning region may include a scanning start position, a scanning end position, and a distance between the main body 923 of the radiation device 922 and the scanning start position. In some embodiments, the control platform 912 may determine the scanning region based on a body portion of the service receiver to be scanned and the image data of the service receiver on the couch 924. The body portion of the service receiver to be scanned may be selected or input by the service provider through the control platform 912. The body portion of the service receiver to be scanned may also be automatically acquired by the control platform 912 according to information of the service receiver (e.g., the identity information). For example, the service receiver may confirm the body portion to be scanned when optical imaging is made. When the service receiver is verified by the identity recognition device, the control platform 912 may acquire the identity information of the service receiver and the corresponding body portion to be scanned. The body portion to be scanned may include the head, the neck, the chest, the abdomen, a leg, or the like, or any combination thereof. After obtaining the body portion to be scanned, the control platform 912 may identify the corresponding body portion according to the image data of the service receiver on the couch 924, and determine that the scanning region includes the body portion to be scanned. In some embodiments, the control platform 912 may further process (e.g., analyze, calculate, etc.) the image data of the service receiver on the couch 924 to determine parameters of the scanning region. For example, the control platform 912 may determine the scanning start position, the scanning end position, and the distance between the main body 923 of the radiation device 922 and the scanning start position by analyzing a relative distance between two reference objects on the image data, a magnification of the image data, etc.

In some embodiments, the control platform 912 may determine, based on the image data (e.g., the image data of the service receiver on the couch 924), a body characteristic of the service receiver. The control platform 912 may determine, based on the body characteristic of the service receiver, the scanning region. The body characteristic may include height, body shape, body proportion, posture (e.g., lying on a back from head to foot, lying on a back from foot to head, lying on a side from head to foot, etc.), etc. Merely by way of example, the control platform 912 may determine, based on the height of the service receiver, the scanning region. Since the physiological structure of the human body has a universality, a regional range of a portion of the human body may have a correspondence with the height of the human body. Therefore, the position and range of the body portion to be scanned may be determined based on the height of the service receiver. For instance, the control platform 912 may calculate the regional range of the body portion to be scanned on the service receiver according to the height of the service receiver and distribution of the body portion to be scanned on the human body with the corresponding height. The control platform 912 may designate the regional range of the body portion to be scanned as the scanning region. In some embodiments, the control platform 912 may also determine scanning parameters based on the body characteristic (e.g., body shape) of the subject. The scan parameters may include a width of a scanning view, a scanning time, a scanning voltage, a scanning current, a scanning direction, or the like.

In some embodiments, after the scanning region is determined, the control platform 912 may control the radiation device 922 to scan the subject according to the scanning region. For example, the control platform 912 may cause the couch 924 to move and cause the main body 923 of the radiation device 922 to perform radiation scanning based on the scanning region. In some embodiments, the control platform 912 may display the determined scanning region to the service provider, and control the radiation device 922 to scan the subject after obtaining a confirmation signal of the service provider. By automatically determining the scanning region, the operation difficulty of the service provider may be effectively reduced, thereby improving the efficiency of the scanning.

In some embodiments, the radiation room 920 may be disposed with a guide device for positioning. The guide device for positioning may be configured to guide the subject to position correctly. In some embodiments, the guide device for positioning may include a display device. The display device may be configured to display guide information. The guide device for positioning may also include an audio device. The audio device may be configured to play the guide information. The display of the guide information may include a video display, a voice display, an image display, a text display, or the like. For example, the guide information may include an image including a target posture or position, a point of a posture or position, a sequence of operations to achieve the target posture or position, etc. In some embodiments, the radiation room 920 may also display other guide information to the service receiver. The other guide information may include information (e.g., ground indicators) that guides the service receiver to a detection location after entering the radiation room 920, information (e.g., a display device) that guides the service receiver to place items that interfere with the scanning (e.g., a coat, jewelry, a mobile phone, etc.), etc. In some embodiments, the service provider may provide voice guidance to the service receiver through a voice device. By setting the guide device for positioning, the service receiver may be quickly and effectively guided to the correct position or posture to reduce the operation difficulty of the service provider, thereby improving the efficiency of the scanning. In addition, the guide may prevent contact between the service receiver and the service provider, thereby avoiding cross-infection between the service receiver and the service provider.

In some embodiments, the guide device for positioning (e.g., the display device) may be operably connected with the control platform 912, such as in an electrical connection, in a wireless connection, etc. The control platform 912 may control the guide device for positioning to display the guide information.

In some embodiments, the control platform 912 may determine, based on the image data, at least one of a posture or a position of the service receiver on the couch 924. For instance, the control platform 912 may determine a current posture and/or a current position of the service receiver according to the image data obtained by the image acquisition device. For example, the current posture and/or the current position of the service receiver may include a lying posture (e.g., lying on the back, lying on the side, etc.), a position of the head, a posture of the hand, a posture of the leg, or the like, or any combination thereof. In some embodiments, the control platform 912 may also acquire the target posture and/or the target position of the service receiver. The target posture may be a preset reference posture or historical posture corresponding to the body portion to be scanned on the service receiver stored in the control platform 912. The target position may be a preset reference position or historical position corresponding to the body portion to be scanned on the service receiver stored in the control platform 912. In some embodiments, the control platform 912 may determine whether the at least one of the posture and/or the position of the service receiver satisfies a condition. For example, the control platform 912 may determine whether the at least one of the posture and/or the position of the service receiver is consistent with the target posture and/or the target position of the service receiver. In response to determining that the at least one of the posture and/or the position of the service receiver does not satisfy the condition, the control platform 912 may provide a reminder for guiding the service receiver to adjust the at least one of the posture and/or the position. The reminder may include a video reminder, a voice reminder, an image reminder, a text reminder, or the like. In some embodiments, the control platform 912 may control the guide device for positioning (e.g., the display device) display the guide information. For example, the guide information may include the image including the target posture or position, the point of the posture and/or position, the sequence of operations to achieve the target posture and/or position, etc.

In some embodiments, the control platform 912 may compare the current posture and/or the current position of the service receiver with the target posture and/or the target position of the service receiver to determine a difference between the current posture and/or the current position of the service receiver and the target posture and/or the target position of the service receiver. The control platform 912 may control the guide device for positioning to display the guide information based on the difference between the current posture and/or the current position of the service receiver and the target posture and/or the target position of the service receiver. For example, if the control platform 912 determines that the difference between the current posture and/or the current position of the service receiver and the target posture and/or the target position of the service receiver is a posture of the hand, that is, the current posture of the hand is inconsistent with the target posture of the hand, the control platform 912 may control the guide device for positioning to display guide information regarding the target posture of the hand. As another example, when the control platform 912 determines that a current posture of the service receiver is lying on the side from foot to head and a target posture of the service receiver is lying on the back from head to foot, the control platform 912 may control the guide device for positioning to display guide information regarding the lying posture of the service receiver. By displaying the guide information regarding the target posture and the target position, the service receiver may position correctly in a short time, thereby improving the efficiency of the scanning.

In some embodiments, the control platform 912 may determine the current posture and/or the current position of the service receiver via a first machine learning model. Exemplary machine learning models may include a neural network model, a decision tree model, a support vector machine, or the like, or any combination thereof. For example, the first machine learning model may be trained through a plurality of sample pairs. Each of the plurality of sample pairs may include image data of a sample service receiver on a couch and at least one of a posture tag and/or a position tag of the sample service receiver.

In some embodiments, the radiation room 920 may include a disinfection device. The disinfection device may be configured to disinfect the radiation room 920 according to one or more disinfection parameters. The disinfection device may include an ultraviolet lamp, an air disinfection machine, a disinfectant spraying device, a plasma disinfection device, or the like, or any combination thereof. In some embodiments, the radiation room 920 may be disposed with more than one disinfection devices.

In some embodiments, the disinfection device may include one or more ultraviolet lamps. The ultraviolet lamp may be mounted on the top wall, the sidewalls, and/or the ground (i.e., the bottom wall) of the radiation room 920. In some embodiments, a count of the ultraviolet lights may be two or more to perform comprehensive disinfection of the space inside the radiation room 920. In some embodiments, the intensity of one of the ultraviolet lamps may be adjustable. The intensity of the ultraviolet lamp may be set according to the type of pathogens. For example, the intensity of the ultraviolet lamp (e.g., an irradiation dose of the ultraviolet light) may be set to exceed 162000 μWs/cm² for the 2019 new coronavirus. In some embodiments, the disinfection device may include a disinfectant spraying device. A type and/or concentration of disinfectant may be adjusted according to the type of pathogens. For example, peracetic acid disinfectant may be used for the 2019 new coronavirus.

In some embodiments, the disinfection device may be turned on or off based on detection information regarding whether there is anyone in the radiation room 920. For example, a body detection device may be disposed in the radiation room 920. The body detection device may include an image recognition device (e.g., a face recognition device, an object recognition device, an imaging sensor, etc.), an infrared sensing device, a pressure sensing device, or the like. The body detection device may be operably connected with the disinfection device. When the body detection device detects that someone is in the radiation room 920, the disinfection device may be turned off. When the body detection device detects that no one is in the radiation room 920, the disinfection device may be automatically opened. In some embodiments, the body detection device (e.g., the infrared sensing device) may determine whether there is anyone in the radiation room 920 by monitoring the person to enter and exit the radiation room 920. In some embodiments, the body detection device (e.g., the image recognition device) may perform a human recognition on images of the radiation room 920 based on an image recognition technique to determine whether there is anyone in the radiation room 920. In some embodiments, the disinfection device may be operably connected with the control platform 912. The control platform 912 may determine whether there is anyone in the radiation room 920 according to the image acquired by the image acquisition device and/or the operation state of the radiation device. For example, when the radiation device is operating, the control platform 912 may determine there is someone in the radiation room 920. The control platform 912 may further control the disinfection device to be turned on or off based on the determination. In the embodiment, the disinfection device may be turned on or off based on the body detection device, which may effectively prevent damage to the human body. In addition, the disinfection device may be turned on in time to improve the operation efficiency of the medical cabin.

In some embodiments, before the disinfection device is turned on, the control platform 912 may provide a reminder for confirming to the service provider. Therefore, an error start of the disinfection device may be prevented.

In some embodiments, the disinfection device may disinfect the radiation room according to one or more disinfection parameters. The disinfection parameters may include a disinfection time, a disinfect intensity, or the like, or any combination thereof. In some embodiments, the one or more disinfection parameters may be determined based on an input instruction of the service provider (e.g., the operator, the doctor, working personnel, an assistant, etc.) in the control room 910. For example, the disinfection device may perform disinfection according to a disinfection instruction of an operator in the control room 910. The disinfection instruction may include a disinfection time and/or a disinfect intensity. The disinfection instruction may be set by the operator through at least one of the control platform 912, a button, a selector, an interaction interface, etc. In some embodiments, the control platform 912 may provide a reminder for disinfecting to the service provider periodically (e.g., every 1 hour, 3 hours, etc.). When the reminder is provided, the control platform 912 may display a disinfection time and a disinfect intensity of recorded last disinfection for the radiation room 920.

In some embodiments, when the service receiver is detected for pathogens infection (e.g., the 2019 new coronavirus, the influenza A virus, etc.), after the detection is completed, the radiation room 920 may be disinfected before receiving a next service receiver, thereby avoiding cross-infection between the service receivers. In some embodiments, the disinfection time of the disinfection device may be a preset time (e.g., 5 minutes, 10 minutes, 15 minutes, etc.). The disinfection intensity of the disinfection device may be a preset intensity (e.g., a preset irradiation dose of the ultraviolet light, a preset disinfectant spraying volume, etc.). In some embodiments, the one or more disinfection parameters (e.g., the disinfection time and/or the disinfecting intensity) of the disinfection device may be determined according to scanning data of the service receiver acquired by the radiation device 922. For example, the control platform 912 may determine a detection result based on the scanning data of the service receiver. When the detection result indicates that the service receiver has a possibility of pneumonia such as bacterial pneumonia, viral pneumonia (e.g., the 2019 new coronavirus, the influenza A virus, etc.), fungal pneumonia, parasitic pneumonia, idiopathic interstitial pneumonia, etc., the control platform 912 may control the disinfection device to disinfect according to a first preset disinfection time (e.g., 20 minutes, 30 minutes, etc.) and/or a first preset disinfection intensity (e.g., a large irradiation dose of the ultraviolet light, a large disinfectant spraying volume, etc.). When the detection result indicates that the service receiver has no possibility or the low possibility of virus infection (e.g., pneumonia), the control platform 912 may control the disinfection device to disinfect according to a second preset disinfection time (e.g., 3 minutes, 5 minutes) and/or a second preset disinfection intensity (e.g., a little irradiation dose of the ultraviolet light, a little disinfectant spraying volume, etc.). Alternatively, the control platform 912 may control the disinfection device not to perform the disinfection. By performing the disinfection based on the detection result of the service receiver, the effect of disinfection may be ensured. In addition, the efficiency of the medical cabin may be improved. In some embodiments, the control platform 912 may determine the detection result of the service receiver using a second machine learning model. Exemplary machine learning models may include a neural network model, a decision tree model, a support vector machine, or the like, or any combination thereof. For example, the second machine learning model may be trained through a plurality of sample pairs. Each of the plurality of sample pairs may include scanning data of a sample service receiver and a tag whether the sample service receiver is infected with pneumonia such as bacterial pneumonia, viral pneumonia (e.g., the 2019 new coronavirus, the influenza A virus, etc.), fungal pneumonia, parasitic pneumonia, the idiopathic interstitial pneumonia, etc.

In some embodiments, the one or more disinfection parameters (e.g., the disinfection time and/or the disinfecting intensity) of the disinfection device may be determined according to other factors. The other factors may include the body shape of the service receiver, the size of the radiation room 920, the temperature of the radiation room 920, the humidity of the radiation room 920, or the like. For example, the control platform 912 may store a disinfection time and/or a disinfection intensity corresponding to each of a plurality of combinations of the factors. The control platform 912 may retrieve a corresponding disinfection time and/or a corresponding disinfection intensity based on the one or more factors. In some embodiments, the control platform 912 may also control a plurality of disinfection devices to perform combined disinfection, to improve the effectiveness of the disinfection and reduce the disinfection time. For example, the ultraviolet lamp and the disinfectant spraying device may be used to disinfect.

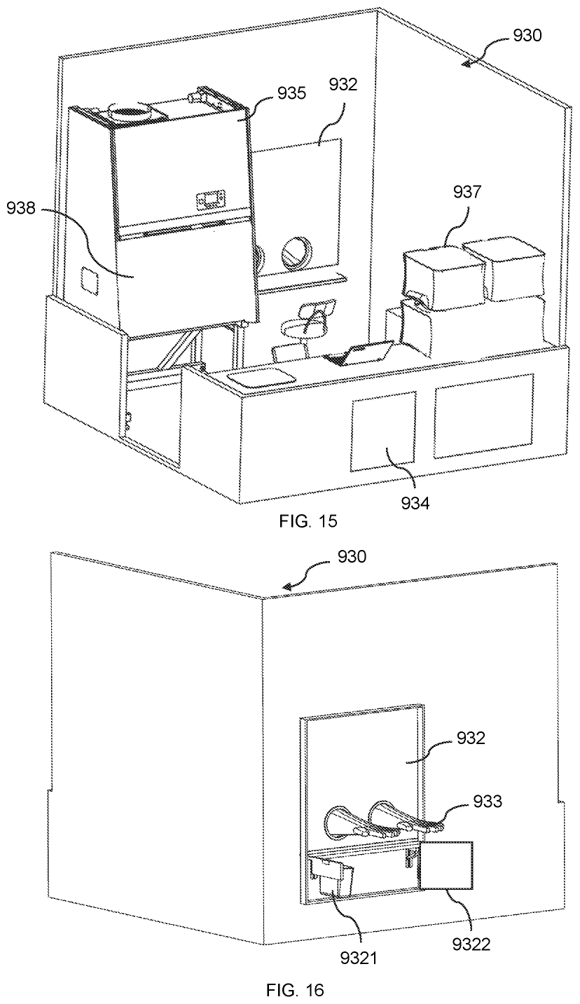

FIG. 14 is a schematic diagram illustrating a sample processing room according to some embodiments of the present disclosure. FIG. 15 is a schematic diagram illustrating an internal structure of a sample processing room according to some embodiments of the present disclosure. FIG. 16 is a schematic diagram illustrating an external structure of a sample processing room according to some embodiments of the present disclosure. In some embodiments, the movable medical device 900 may further include a sample processing room 930. The sample processing room 930 in some embodiment of the present disclosure may be described below with reference to FIGS. 14-16. It should be noted that the descriptions of the sample processing room are merely provided for illustration, and not intended to limit the scope of the present disclosure.

In some embodiments, the sample processing room 930 may include a sampling chamber and a sample detection chamber. The sampling chamber may be configured to provide a sampling service. The sample detection chamber may be configured to provide a sample detection service. Alternatively, the sampling chamber and the sample detection chamber may be integrated into the same room (i.e., a sampling and sample detection chamber). The sampling and sample detection chamber may be configured to provide a sampling service and a sample detection service.

In some embodiments, as shown in FIG. 14, the sample processing room 930 may be disposed with a pressure adjustment device 940. The pressure adjustment device 940 may be configured to adjust a gas pressure inside the sample processing room 930 to be lower than a gas pressure outside the sample processing room 930. Therefore, the leakage of pathogens in the sample processing room 930 may be effectively prevented. In some embodiments, a portion of components (e.g., a suction port) of the pressure adjustment device 940 may be disposed inside the sample processing room 930. Remaining components (e.g., a pumping device, an air-purifying device, an exhaust port, etc.) of the pressure adjustment device 940 may be disposed outside the sample processing room 930.

In some embodiments, as shown in FIG. 14, the sample processing room 930 may include a buffer chamber 939. The buffer chamber 939 and the sampling and sample detection chamber (or the sampling chamber, and the sample detection chamber) may be connected through a first closable gate 9392. When a service provider (e.g., an operator, medical staff, etc.) enters the sample processing room 930 from the third door 931, the service provider may replace a protection article (e.g., protective clothing, a protection mask, a mask, etc.) in the buffer chamber 939. Subsequently, the service provider may enter the sampling and sample detection chamber (or the sampling chamber and the sample detection chamber) through the first closable gate 9392. When the service provider exits the sample processing room 930, a service provider may also pass through the buffer chamber 939. The service provider may perform operations, such as a body disinfection, a replacement of the protection article, etc., in the buffer chamber 939. In some embodiments, the buffer chamber 939 may be disposed with a disinfection device 9391 (e.g., a disinfection pan) for disinfecting the protection article. The disinfection device 9391 may be the same as or similar to the disinfection device in the radiation room 920.

In some embodiments, as shown in FIGS. 14-16, a sampling window 932 may be disposed in the sample processing room 930. The sampling window 932 may be used for an interaction between a service provider (e.g., medical staff, etc.) and a service receiver (e.g., a patient, an infected person, etc.) of the sampling service. Isolation gloves 933 may be disposed on the sampling window 932. The service provider in the sample processing room 930 may sample the service receiver to be detected outside the sample processing room 930 through the isolation gloves 933. For example, the service provider in the sample processing room 930 may sample a throat swab sample, a nasopharyngeal swab sample, an oropharyngeal swab sample, a sample of a bronchoalveolar lavage fluid, a biopsy specimen of a lung, a sputum specimen, a blood sample, or the like, or any combination thereof, of the service receiver to be detected through the isolation gloves 933. The isolation gloves 933 may be a glove made of rubber or other materials. The isolation gloves 933 may be configured to isolate pathogens. In some embodiments, to facilitate the operation of the service provider, a first hanging basket 9321 and a second hanging basket 9322 may be further disposed outside the sampling window 932. In some embodiments, the first hanging basket 9321 and the second hanging basket 9322 may be disposed at a position that can be reached by the service provider through the isolation gloves 933. The first hanging basket 9321 and the second hanging basket 9322 may be used to store a sampling device (e.g., a sample tube) and the sample of the service receiver, respectively. By setting the sampling window 932 and the isolation gloves 933, the service provider may conveniently sample the service receiver to be detected. In addition, the contact between the service provider and the service receiver to be detected may be avoided, thereby reducing cross-infection between the service provider and the service receiver to be detected. In some embodiments, a robotic system may be disposed on the sampling window 932. The robotic system may be configured to sample by controlled by the service provider (e.g., the doctor, the operator, etc.) or based on a sample instruction. The robotic system may improve the sampling efficiency and ease the strain on medical resources. In addition, using the robotic system may reduce the infection risk of the service provider.

In some embodiments, as shown in FIGS. 14-16, a transmission channel 934, an inactivation device 938, a biosafety cabinet 935, a refrigerator 936, a sample analysis device 937, etc., may be disposed in the sample processing room 930, so that the sample processing room 930 may be configured to detect the sample of the service receiver to be detected. The transmission channel 934 may be configured to transfer the sample between the outside of the medical cabin and the sample processing room 930. In some embodiments, a disinfection device (e.g., the ultraviolet lamp, the disinfectant spraying device, etc.) may be disposed in the transmission channel 934 to disinfect the transferred sample. For example, a service provider (e.g., an assistant) may transfer the sample to a disinfection region of the disinfection device through the transmission channel 934. The inactivation device 938 may be configured to inactivate the sample to prevent infection. For example, the inactivation device 938 may include a high-temperature inactivation device. The high-temperature inactivation device may heat the sample to a temperature higher than a temperature threshold (e.g., 50 degrees, 56 degrees, 60 degrees, 65 degrees, 70 degrees, etc.). The biosafety cabinet 935 may be configured to provide a safe environment for storing the sample to prevent leakage of the sample. In some embodiments, the biosafety cabinet 935 may be disposed with a pressure adjustment device. The pressure adjustment device may be configured to adjust a gas pressure inside the biosafety cabinet 935 to be lower than a gas pressure outside the biosafety cabinet 935 to prevent the leakage of pathogens, thereby protecting the safety of the service provider. The sample analysis device 937 may be used for sample detection to determine whether the service receiver to be detected is infected with pathogens. For example, the sample analysis device 937 may determine whether the service receiver to be detected is infected with the 2019 novel coronavirus according to the detection and analysis of the sample.

An exemplary sample detection process may include placing the sample in the transmission channel from the outside of the transmission channel; using the disinfection device (e.g., an ultraviolet lamp, an alcohol spraying device, etc.) in the transmission channel to disinfect the sample, removing the from the inside of the transmission channel by the service provider in the sample processing room; transferring the sample to the biosafety cabinet, and using the inactivation device to inactivate the sample, and detecting the inactivated sample by using the sample analysis device. The refrigerator 936 in the sample processing room 930 may be configured to store medical articles, such as a detection reagent, etc. In some embodiments, the sample may also be stored in a safety box and transferred to the sample processing room 930 by the service provider through an access channel of the service provider. For example, the service provider (e.g., a transferrer) may transfer the safety box to the sample processing room 930 through the access channel. The safety box may include a sealable device, thereby preventing the sample in the safety box from contacting the outside during the transfer.

In some embodiments, when the sample processing room 930 is a sampling room, the sample detection may be performed in other places (e.g., a clinical laboratory of a hospital). In some embodiments, when the sample processing room 930 is a sample detection room, the sampling may be performed outside the medical cabin (e.g., a temporary sampling point). In some embodiments, when the sample processing room 930 is a sampling and sample detection room, the sample sampled through the sampling window may be placed in the transmission channel with the assistance of the service provider (e.g., an assistant, a volunteer, etc.).

In some embodiments, after the sample of the service receiver to be detected is detected, the service provider may input a sample detection result of the service receiver to be detected into the movable medical device 900 (e.g., a database). For example, the sample detection result may be saved to a local terminal, uploaded to a remote server, sent to the control platform 912, etc. In some embodiments, the control platform 912 may obtain the sample detection result corresponding to the service receiver. Further, the control platform 912 may determine a final detection result of the service receiver according to the sample detection result and the scanning data of the service receiver. For example, for the 2019 novel coronavirus, the final detection result of the service receiver may include a patient with the novel coronavirus pneumonia, a person infected with the novel coronavirus, a non-infected person, etc.

In some embodiment of the present disclosure, the movable medical device 900 may be configured to provide the medical imaging service, the sampling service, the sample detection service, etc. The service receiver to be detected (or scanned) may be scanned and sampled via the movable medical device 900. The sample of the service receiver may be detected via the movable medical device 900. The sample detection result and the scanning data may be comprehensively analyzed and processed. The final detection result of the service receiver to be detected (or scanned) may be quickly determined. For the detection of the 2019 novel coronavirus, the movable medical device 900 in the embodiment of the present disclosure may quickly and accurately screen out pneumonia patients and virus-infected persons. Therefore, corresponding measures (e.g., the isolation, the treatment, etc.) may be performed in time to effectively prevent a further expansion of the virus.

It should be noted that the description of the movable medical device 900 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the medical cabin of the movable medical device 900 may include other rooms configured to perform other functions, such as performing a treatment, dispensing medical items, etc. As another example, the movable medical device 900 may include a plurality of movable platforms. Each of the plurality of movable platforms may be caused to move a portion of the medical cabin. As still another example, the movable medical device 900 may include no movable platform. The medical cabin may be moved by an external movable platform. As still another example, the movable medical device 900 may include a monitoring platform and an energy platform.

FIG. 17 is a block diagram illustrating an exemplary power supply system according to some embodiments of the present disclosure.

As shown in FIG. 17, the power supply system 1700 may include electric source systems 1720, and electronic components 1740. The electronic components 1740 may be electrically connected through one or more printed circuit boards (PCB). The connected electronic components 1740 may form different modules for performing different functions, such as controlling, monitoring, and/or adjusting a voltage provided by one of the electric source systems 1720 to a load. For example, different modules may include an acquisition module 1742, a control module 1744, an adjustment module 1746, and a display module 1748. In some embodiments, a module may be a hardware circuit composed of one or more electronic components. In some embodiments, each module may be implemented as a PCB including connected electronic components. In some embodiments, the acquisition module 1704, the control module 1744, the monitoring and processing module 1746, and the display module 1748 may be integrated into one single PCB. In some embodiments, the modules may also be implemented as an application or set of instructions read and executed by a processor. Further, the modules may be any combination of the hardware circuits and the application/instructions. For example, the modules may be part of the processing device 120 when the processing device 120 is executing the application/set of instructions.

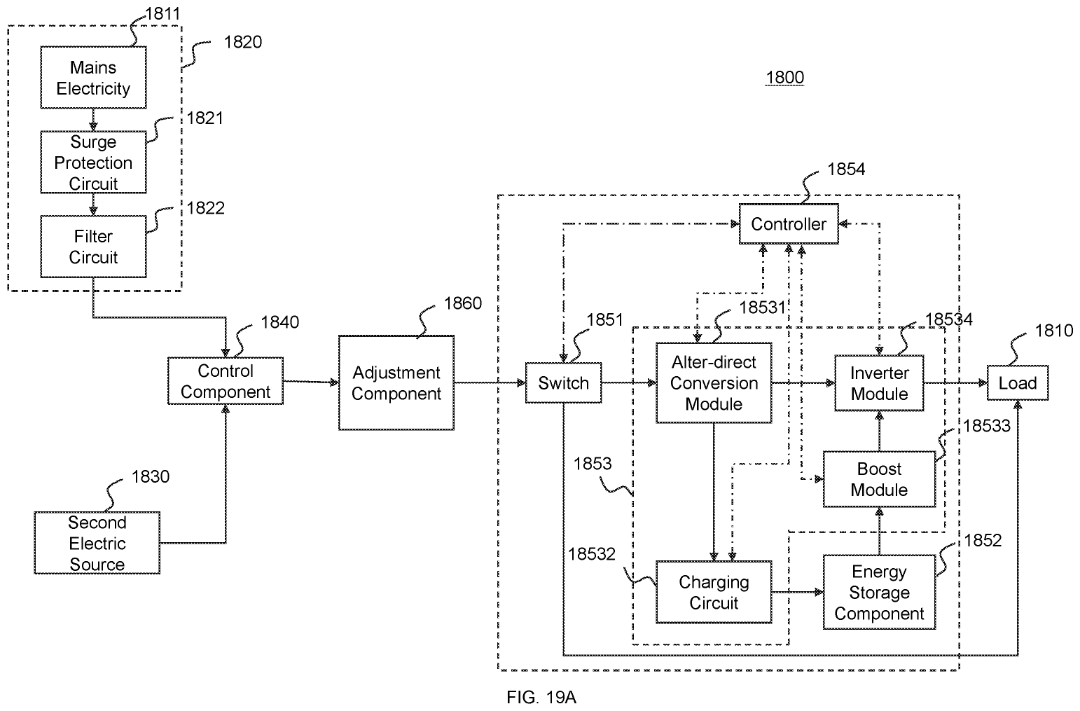

The electric source systems 1720 may be configured to provide power to a load of the movable medical device. The electric source systems 1720 may provide power through multiple electric source systems (e.g., a first electric source system, a second electric source system, a third electric source system, etc.) of a power supply apparatus. More descriptions for the multiple electric source systems may be found elsewhere in the present disclosure (e.g., FIGS. 18-20 and the descriptions thereof).

The acquisition module 1742 may be configured to obtain data associated with one or more parameters (e.g., a voltage, a current, etc.) of each of components in the power supply system 1700 or a load powered by the power supply system 1700. The obtained data may be transmitted to the control module 1744, the adjustment module 1746, and/or the display module 1748 for further processing.

The control module 1744 may be configured to control an operation of one or more components in the power supply system 1700. In some embodiments, the control module 1744 may determine an operation state of at least one of the first electric source system or the second electric source system based on the acquired data of the acquisition module 1742, and control an operation of the at least one of the first electric source system or the second electric source system based on the operation state. For example, in response to determining that the first electric source system does not include an anomaly, the control module 1744 may control the first electric source system to provide power for the load, i.e., an electrical conductance between the first electric source system and the load, such that the first electric source system may power the load. The control module 1744 may control an electrical conductance between the first electric source system and the adjustment module (e.g., the adjustment component 1860 as described in FIG. 18). In response to determining that the first electric source system includes an anomaly, the control module 1744 may control the second electric source system to provide power for the load, i.e., an electrical conductance between the second electric source system and the load. The control component 1744 may control an electrical conductance between the second electric source system and the adjustment module (e.g., the adjustment component 1860 as described in FIG. 18), such that the second electric source system may power the load.

The adjustment module 1746 may be configured to adjust an input voltage of the load and/or output voltage of one of the electric source systems 1720. In some embodiments, in response to determining that the first electric source system does not include the anomaly, the adjustment module 1746 may acquire information associated with an output voltage (or current) of the first electric source system (or the control module) and/or the input voltage (or current) of the adjustment module 1746 (e.g., the adjustment component 1860 as described in FIG. 18) when the first electric source system provides power for the load. In response to determining that the first electric source system includes an anomaly, the adjustment module 1746 may acquire information associated with an output voltage (or current) of the second electric source system (or the control module) and/or the input voltage (or current) of the adjustment module 1746 (e.g., the adjustment component 1860 as described in FIG. 18) when the second electric source system provides power for the load.

In some embodiments, the control module 1744 may be configured to control the adjustment module 1746 (e.g., the adjustment component 1860 as described in FIG. 18) to adjust the input voltage of the load and/or output voltage of one of the electric source systems 1720. In some embodiments, the control module 1744 may be configured to control one of the electric source systems 1720 to adjust the input voltage of the load and/or output voltage of one of the electric source systems 1720. For example, the control module 1744 may determine whether the output voltage (or current) of the first electric source system (or the control module 1744) and/or the input voltage (or current) of the adjustment module 1746 (e.g., the adjustment component 1860 as described in FIG. 18) is equal to a preset voltage value or in a preset voltage range. In response to determining that the output voltage (or current) of the first electric source system (or the control module 1744) and/or the input voltage (or current) of the adjustment module 1746 (e.g., the adjustment component 1860 as described in FIG. 18) is not equal to the preset voltage value or not in the preset voltage range, the control module 1744 may control the adjustment module 1746 (e.g., the adjustment component 1860 as described in FIG. 18) to adjust the input voltage (or current) of the adjustment module 1746 and/or output voltage of one of the electric source systems 1720 to be equal to the preset voltage value or in the preset voltage range, such that the output voltage (or current) of the adjustment module 1746 or the input voltage of the load satisfies a condition (e.g., equal to the preset voltage value or in the preset voltage range).

The display module 1748 may be configured to display information regarding providing power, such as the operation state of at least one of the first electric source system or the second electric source system, whether a remaining capacity of an electric source is less than an energy threshold, feedback for adjusting the output voltage of the electric source, etc.

In some embodiments, the control module 1744 may be configured to control the display module 1748 to display information regarding providing power.

It should be noted that the above description of the processing device 120 is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. In some embodiments, one or more modules may be combined into a single module. For example, the control module 1744 and the adjustment module 1746 may be combined into a single module. The acquisition module 1742 and the control module 1744 may be integrated into one single module. In some embodiments, one or more modules may be added or omitted in the processing device 120. For example, the storage module may be added. As another example, the adjustment module 1746 may be divided into a monitoring module and an adjusting module.

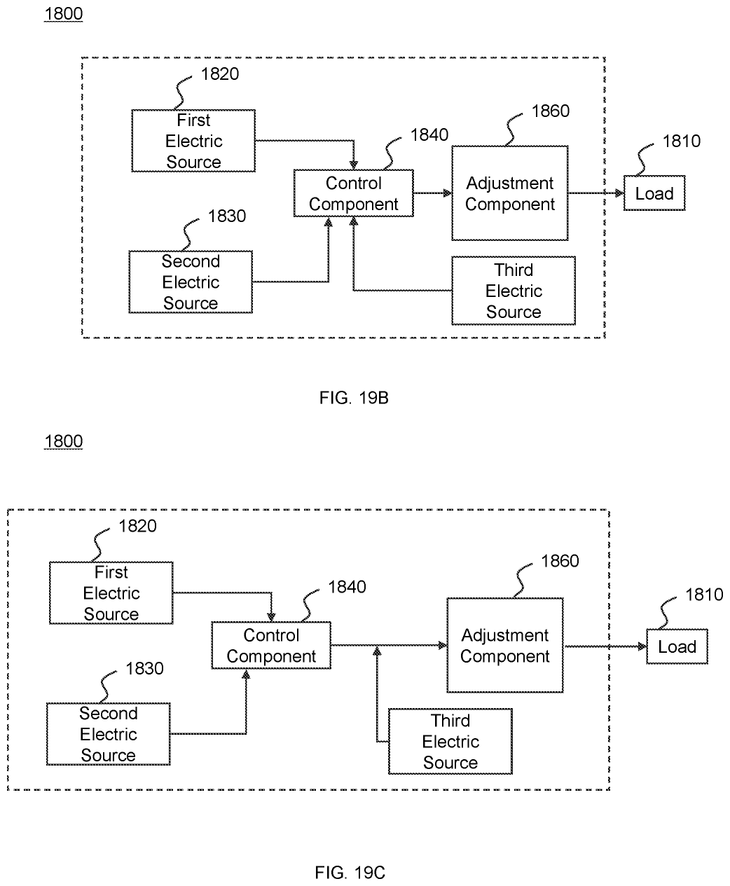

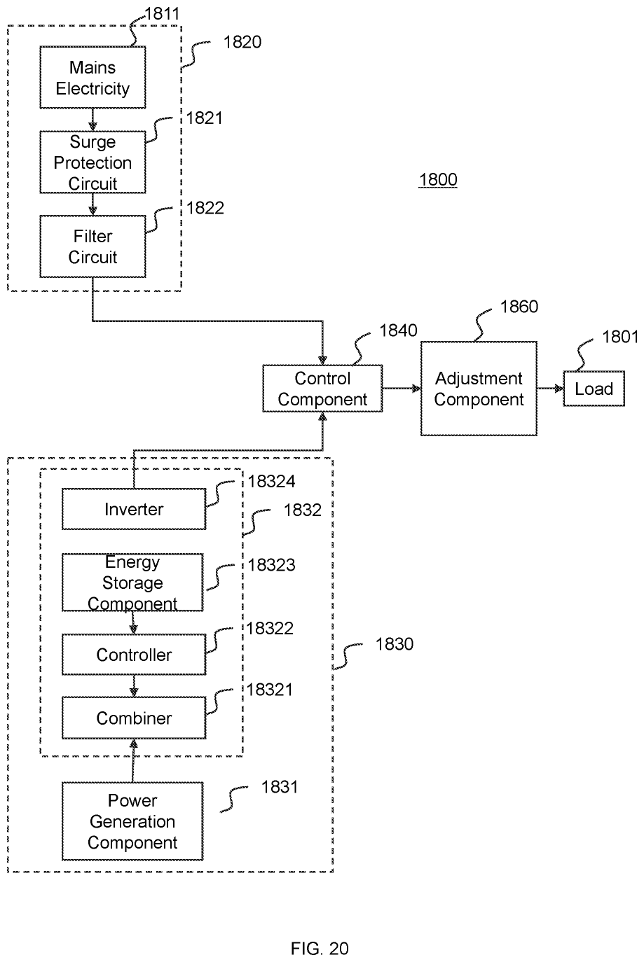

FIG. 18 is a schematic diagram illustrating an exemplary power supply system according to some embodiments of the present disclosure. FIGS. 19A-C are schematic diagrams illustrating an exemplary embodiment of the power supply system to some embodiments of the present disclosure. FIG. 20 is a schematic diagram illustrating another exemplary embodiment of the power supply system according to some embodiments of the present disclosure.

In some embodiments, a power supply system 1800 may be configured to provide power to a load 1810. The load 1810 may refer to an electric device. In some embodiments, the load 1810 may include one or more components in a movable medical device that includes a medical cabin, a movable platform, a monitoring platform, etc. More descriptions for the movable medical device may be found elsewhere in the present disclosure (e.g., FIGS. 2-16). For example, the medical cabin may be configured to accommodate medical resources for providing one or more types of medical services. The movable platform may be configured to move the medical cabin to a desired location. It should be noted that the descriptions of the power supply apparatus are merely provided for illustration, and not intended to limit the scope of the present disclosure.

In some embodiments, the load 1810 may include a medical device (e.g., a positron emission tomography (PET) device, a single-photon emission computed tomography (SPECT) device, a computed tomography (CT) device, a magnetic resonance imaging (MRI) device, a radiation therapy (RT) device, etc.), one or more components for adjusting operation state of at least one of the components of the movable medical device or environment parameters where the medical cabin is located (e.g., the temperature adjustment device 351, the humidity adjustment device 352, etc.), one or more components for sampling and sample detection (e.g., the inactivation device 938, the biosafety cabinet 935, the refrigerator 936, the sample analysis device 937, etc.), or the like, or any combination thereof.

As shown in FIG. 18, the power supply system may include a first electric source system 1820, a second electric source system 1830, a control component 1840, and an adjustment component 1860. The control component 1840 may be electrically connected with the first electric source system 1820 and the second electric source system 1830, respectively. The adjustment component 1860 may be electrically connected with the load 1810 and the control component 1840. As used herein, an “electrical connection” between two different components may be understood as a connection between the two components through physical lines (e.g., PCB copper foil or wires) that can transmit electrical signals.

The first electric source system 1820 and the second electric source system 1830 may be configured to provide power to the load 1810. The first electric source system 1820 may include an energy storage component (e.g., the energy storage component 342, the energy storage component 543, etc.), a mains electricity, a power generation component (e.g., the power generation component 341, the power generation component 544), or the like. The second electric source system 1830 may include an energy storage component, a power generation component, or the like.

The control component 1840 may be configured to determine an operation state of at least one of the first electric source system 1820 or the second electric source system 1830, and control an operation of the at least one of the first electric source system or the second electric source system based on the operation state and determine one of the first electric source system 1820 and the second electric source system 1830 to provide power for the load 1810 based on the operation state. The operation state of an electric source (e.g., the first electric source system 1820 or the second electric source system 1830) may indicate whether the electric source includes an anomaly. In some embodiments, the operation state of an electric source (e.g., the first electric source system 1820 or the second electric source system 1830) may include a normal state (i.e., the electric source includes no anomaly) and an abnormal state (i.e., the electric source includes an anomaly). The normal state may indicate that an output voltage and/or an output current of the electric source such that the output voltage and/or the output current of the electric source may be supplied to a load (e.g., the load 1810). The normal state of an electric source may be that at least one of the output voltage or the output current of the electric source satisfies a first condition. The first condition may be that the output voltage is in a proper range (e.g., from a first voltage threshold to a second voltage threshold), the output current is in a proper range (e.g., from a first current threshold to a second current threshold), a change value of the output voltage in a specific period is less than a voltage change threshold, a change value of the output current in a specific period is less than a current change threshold, the electric source is in a -full-phase state, etc.

The abnormal state of an electric source may indicate that at least one of the output voltage or the output current of the electric source includes an anomaly such that the output voltage and/or the output current of the electric source cannot be supplied to a load (e.g., the load 1810). The abnormal state of an electric source may be that at least one of the output voltage or the output current of the electric source satisfies a second condition. The second condition may be that the output voltage is not in a proper range (e.g., less than a first voltage threshold (e.g., the output voltage is 0) or exceeding a second voltage threshold), the output current is not in a proper range (e.g., less than a first current threshold or exceeding a second current threshold), a change value of the output voltage in a specific period exceeds a voltage change threshold, a change value of the output current in a specific period exceeds a current change threshold, the electric source is phase-lacking, etc.

For example, the first electric source system 1820 may be in an abnormal state if the first electric source system 1820 outputs no current. As another example, the first electric source system 1820 may be in an abnormal state if the output voltage of the first electric source system 1820 may be overvoltage or undervoltage. As still another example, the first electric source system 1820 may be in an abnormal state if the output voltage and/or the output current of the first electric source system 1820 may be unstable. As still another example, the first electric source system 1820 may be in an abnormal state if the first electric source system 1820 may include a default phase.

In some embodiments, the control component 1840 may be configured to determine whether the first electric source system 1820 includes an anomaly. In response to determining that the first electric source system 1820 does not include an anomaly, the control component 1840 may control the first electric source system 1820 to provide power for the load 1810, i.e., an electrical conductance between the first electric source system 1820 and the load 1810. In some embodiments, the control component 1840 may control an electrical conductance between the first electric source system 1820 and the adjustment component 1860, such that the adjustment component 1860 may acquire information associated with an output voltage (or current) of the first electric source system 1820 (or the control component 1840) and/or the input voltage (or current) of the load 1810 when the first electric source system 1820 provides power for the load 1810. A used herein, “electrical conductance” between different components refers to that electrical signals can be transmitted between the different components. In response to determining that the first electric source system 1820 includes an anomaly, the control component 1840 may control the second electric source system 1830 to provide power for the load 1810, i.e., an electrical conductance between the second electric source system 1830 and the load 1810. In some embodiments, the control component 1840 may control an electrical conductance between the second electric source system 1830 and the adjustment component 1860, such that the adjustment component 1860 may acquire information associated with an output voltage (or current) of the second electric source system 1830 (or the control component 1840) and/or the input voltage (or current) of the load 1810 when the second electric source system 1830 provides power for the load 1810. Accordingly, the power supply system 1800 may continuously supply power to the load 1800 (e.g., a medical device) by switching multiple electric source systems (e.g., the first electric source system 1820, the second electric source system 1830, etc.), which may effectively prevent the load 1800 (e.g., a medical device) from being powered off.

In some embodiments, the control component 1840 may be configured to detect whether there is a voltage at an interface between the control component 1840 and the electric source (e.g., the first electric source system 1820, the second electric source system 1830, etc.). When there is a voltage at an interface between the control component 1840 and the first electric source system 1820, the control component 1840 may automatically conduct the first electric source system 1820 with the load 1810, such that the first electric source system 1820 may power the load 1810. When there is no voltage at an interface between the control component 1840 and the first electric source system 1820, and there is a voltage at an interface between the control component 1840 and the second electric source system 1830, the control component 1840 may automatically conduct the second electric source system 1830 with the load 1810, such that the second electric source system 1830 may power the load 1810.

In some embodiments, the adjustment component 1860 may be configured to adjust an input voltage of the load 1810 when one of the first electric source system 1820 and the second electric source system 1830 powers the load 1810. The input voltage of the load 1810 may be the same as or close to an output voltage of the adjustment component 1860. For example, the adjustment component 1860 may monitor an output voltage of the control component 1840. The output voltage of the control component 1840 may be the same as or close to an input voltage of the control component 1840, or an input voltage of the adjustment component 1860, or an output voltage of the first electric source system 1820 or the second electric source system 1830. The adjustment component 1860 may determine whether the output voltage of the control component 1840 (or the input voltage of the control component 1840, or the input voltage of the adjustment component 1860, or the output voltage of the first electric source system 1820 or the second electric source system 1830) is equal to a preset voltage value or a preset voltage range from a minimum voltage value to a maximum voltage value. The preset voltage value or the preset voltage range may be determined according to an operation voltage of the load 1810. For example, the preset voltage value may be equal to the operation voltage of the load 1810. As another example, the preset voltage value may be a value within a range around the operation voltage of the load 1810. For instance, a difference between the preset voltage value and the operation voltage of the load 1810 may be less than 1 volt, 2 volts, 3 volts, 4 volts, 5 volts, etc. As still another example, a difference between the minimum voltage value (or the maximum voltage value) and the operation voltage of the load 1810 may be less than 1 volt, 2 volts, 3 volts, 4 volts, 5 volts, etc. If the output voltage of the control component 1840 (or an input voltage of the control component 1840, or the input voltage of the adjustment component 1860, or the output voltage of the first electric source system 1820 or the second electric source system 1830) is equal to the preset voltage value or the preset voltage range, the adjustment component 1860 may directly output the output voltage of the control component 1840. If the output voltage of the control component 1840 (or the input voltage of the control component 1840, or the input voltage of the adjustment component 1860, or the output voltage of the first electric source system 1820 or the second electric source system 1830) is not equal to the preset voltage value or not in the preset voltage range, the adjustment component 1860 may adjust the input voltage of the adjustment component 1860 to the preset voltage value or in the preset voltage range. For example, when the output voltage of the control component 1840 is higher than the preset voltage value of the load 1810, the adjustment component 1860 may decrease the voltage inputted into the adjustment component 1860, such that the output voltage of the adjustment component 1860 is equal to the preset voltage value of the load 1810. As another example, when the output voltage of the control component 1840 is lower than the preset voltage value of the load 1810, the adjustment component 1860 may increase the voltage inputted into the adjustment component 1860, such that the output voltage of the adjustment component 1860 is equal to the preset voltage value of the load 1810. Accordingly, the load 1810 may operate normally when the input voltage of the load 1810 is stable and suitable, thereby prolonging the service life of the load 1810. Therefore, the adjustment component 1860 may ensure the stability of the input voltage of the load 1810, and further ensure the normal operation of the load 1810.

In some embodiments, the adjustment component 1860 may provide feedback for adjusting the output voltage of the electric source (e.g., the first electric source system 1820, the second electric source system 1830, etc.) for powering the load 1860. The feedback may include a prompt instruction and/or an adjustment instruction for adjusting the output voltage of the electric source (e.g., the first electric source system 1820, the second electric source system 1830, etc.). For example, when the output voltage of the control component 1840 (or the output voltage of the adjustment component 1860, or the input voltage of the load 1810) is higher than the preset voltage value of the load 1810, the adjustment component 1860 may provide the feedback for decreasing the output voltage of the electric source (e.g., the first electric source system 1820, the second electric source system 1830, etc.) to the preset voltage value of the load 1810. As another example, when the output voltage of the control component 1840 (or the output voltage of the adjustment component 1860, or the input voltage of the load 1810) is lower than the preset voltage value of the load 1810, the adjustment component 1860 may provide the feedback for increasing the output voltage of the electric source (e.g., the first electric source system 1820, the second electric source system 1830, etc.) to the preset voltage value of the load 1810.

In some embodiments, the control component 1840 may be implemented through a first circuit (also referred to as a control circuit), and the adjustment component 1860 may be implemented through a second circuit (also referred to as a monitoring and adjustment circuit). In some embodiments, the first circuit and the second circuit may be integrated into a circuit that may be configured to implement functions of the control component 1840 and the adjustment component 1860. For example, the first circuit and the second circuit may be integrated into one single printed circuit board.

Referring to FIGS. 19A-19C, in some embodiments, the power supply apparatus 1810 may further include a third electric source system 1850. The third electric source system 1850 may be configured to provide power for the load 1810 in response to determining that each of the first electric source system 1820 and the second electric source system 1830 includes an anomaly. In some embodiments, as shown in FIG. 19A, the third electric source system 1850 may be electrically connected with the adjustment component 1860 and the load 1810. In some embodiments, as shown in FIG. 19B, the third electric source system 1850 may be electrically connected with the control component 1840. In some embodiments, as shown in FIG. 19C, the third electric source system 1850 may be electrically connected with the control component 1840 and the adjustment component 1860, i.e., the third electric source system 1850 may be located between the control component 1840 and the adjustment component 1860. In some embodiments, in response to determining that each of the first electric source system 1820 and the second electric source system 1830 includes an anomaly, the control component 1840 may control an electrical conductance between the load 1810 and the third electric source system 1850. In some embodiments, in response to determining that each of the first electric source system 1820 and the second electric source system 1830 includes an anomaly, the control component 1840 may control an electrical conductance between the third electric source system 1850 and the adjustment component 1860.

In some embodiments, when the first electric source system 1820 or the second electric source system 1830 provides power for the load 1810, the power supply system 1800 may provide power for the load 1810 through the first electric source system 1820 or the second electric source system 1830. At the same time, the first electric source system 1820 or the second electric source system 1830 may provide power for the third electric source system 1850. When the first electric source system 1820 or the second electric source system 1830 provides no power for the load 1810, the power supply system 1800 may provide power for the load 1810 through the third electric source system 1850. The power supply system 1800 may increase the count (number) of electric source systems and control a plurality of electric source systems to supply power in an orderly manner, thereby reducing the probability of power failure of the load 1810.

In some embodiments, to ensure that the load 1810 (e.g., the radiation device, the sample analysis device, etc.) is continuously powered, the third electric source system 1850 may include an uninterruptible power source (UPS). Exemplary UPSs may include an online UPS, a line-interactive UPS, a standby UPS, or the like, or any combination thereof.

In some embodiments, the third electric source system 1850 may be configured for energy storage. Merely by way of example, the third electric source system 1850 may include an energy storage component. When the first electric source system 1820 or the second electric source system 1830 provides power for the load 1810, and the third electric source system 1850 includes no anomaly, the third electric source system 1850 may be electrically connected with the adjustment component 1860. Therefore, the first electric source system 1820 or the second electric source system 1830 may be electrically connected with the third electric source system 1850 through the control component 1840 and the adjustment component 1860.

In some embodiments, a remaining capacity of the third electric source system 1850 may be determined. When the remaining capacity of the third electric source system 1850 is less than a capacity threshold, the third electric source system 1850 may be powered by the first electric source system 1820 or the second electric source system 1830. The third electric source system 1850 cannot provide power for the load 1810 when the remaining capacity of the third electric source system 1850 is less than the capacity threshold. In some embodiments, the capacity threshold may be determined according to a full capacity of the third electric source system 1850, such as 40%, 50%, 60%, 70%, etc., of the full electric energy of the third electric source system 1850. The full capacity of the third electric source system 1850 refers to a maximum capacity that the third electric source system 1850 is derived.

In some embodiments, when the remaining capacity of the third electric source system 1850 is less than the capacity threshold, and the first electric source system 1820 does not include an anomaly, the first electric source system 1820 may be conducted with the load 1810, and the first electric source system 1820 may be conducted with the third electric source system 1850, such that the first electric source system 1820 may power the load 1810 and the third electric source system 1850 (e.g., the energy storage component). The third electric source system 1850 (e.g., the energy storage component) may be controlled to be charged.

In some embodiments, when the remaining capacity of the third electric source system 1850 is less than the capacity threshold, the first electric source system 1820 includes an anomaly, the second electric source system 1830 does not include an anomaly, the control component 1830 may control a conductance between the second electric source system 1830 and the load 1810 and/or block the conductance between the first electric source system 1820 and the load 1810. The control component 1830 may control a conductance between the second electric source system 1830 and the third electric source system 1850, such that the second electric source system 1830 may power the load 1810 and the third electric source system 1850 (e.g., the energy storage component).

When the remaining capacity of the third electric source system 1850 is less than the capacity threshold, and the first electric source system 1820 and the second electric source system 1830 do not include an anomaly, one of the first electric source system 1820 and the second electric source system 1830 may power the load 1810 and another of the first electric source system 1820 and the second electric source system 1830 may power the third electric source system 1850 (e.g., the energy storage component).

In some embodiments, the third electric source system 1850 may be configured to adjust the input voltage of the load 1810.

As shown in FIG. 19A, the third electric source system 1850 may include a switch 1851, an energy storage component 1852 (also referred to as a first energy storage component 1852), a current adjustment component 1853, and a controller 1854. The current adjustment component 1853 may be connected with the energy storage component 1852. The switch 1851 may be connected with the adjustment component 1860, the load 1810, and the current adjustment component 1853. The controller 1854 may be connected with the current adjustment component 1853 and the switch 1851. In some embodiments, the controller 1854 may be configured to determine whether a voltage is inputted into the switch 1851. In response to the determination result that no voltage is inputted into the switch 1851, the controller 1854 may control the energy storage component 1852 to power the load 1810 through the current adjustment component 1853. For example, the controller 1854 may control the switch 1851 to be directly conducted with the current adjustment component 1853 or the energy storage component 1852, such that the energy storage component 1852 may power the load 1810. In response to the determination result that the voltage is inputted into the switch 1851, the controller 1854 may control the switch 1851 to be directly conducted with the load 1810. That is, the first electric source system 1820 or the second electric source system 1830 may power the load 1851.

In some embodiments, the controller 1854 may be configured to obtain the operation state of at least one of the first electric source system 1820 or the second electric source system 1830. The controller 1854 may control the energy storage component 1852 to power the load 1810 based on the operation state. In response to determining that each of the first electric source system 1820 and the second electric source system 1830 includes the anomaly, the controller 1854 may control the energy storage component 1852 to power the load 1810 directly or through the current adjustment component 1853. In response to determining that each of the first electric source system 1820 and the second electric source system 1830 does not include the anomaly, the controller 1854 may control the switch 1851 to be directly conducted with the load 1810.

In some embodiments, the energy storage component 1852 may include a battery or a power storage device. For example, the energy storage component 1852 may include a lithium battery, a lead-acid battery, a supercapacitor, etc.

In some embodiments, the controller 1854 may be further configured to determine an operation state of at least one of the third electric source system 1850 and adjust the input voltage of the load 1810 to satisfy voltage requirements of the load 1810.

In some embodiments, a current of the load 1810 may be an alternating current (AC). When the load 1810 is powered by the third electric source system 1850, an output current of the energy storage component 1852 may be a direct current (DC). Therefore, the output current of the energy storage component 1852 may be converted to an AC to provide power for the load 1810.

In some embodiments, when one of the first electric source system 1820 and the second electric source system 1830 does not include the anomaly, and the energy storage component 1852 needs to be powered, the controller 1854 may control an electrical conductance between the adjustment component 1860 and the current adjustment component 1853 through the switch 1851. The controller 1854 may control an electrical conductance between the first electric source system 1820 or the second electric source system 183 and the load 1810 and an electrical conductance between the first electric source system 1820 or the second electric source system 183 and the energy storage component 1852 through the current adjustment component 1853. The energy storage component 1852 may store the power.

In some embodiments, when one of the first electric source system 1820 and the second electric source system 1830 does not include the anomaly, and the energy storage component 1852 needs no power, the controller 1854 may control an electrical conductance between the switch 1851 and the load 1810. The first electric source system 1820 or the second electric source system 183 may provide power to the load 1810 through the control component 1840, the adjustment component 1860, and the switch 1851.

In some embodiments, the controller 1854 may be connected with the switch 1851. The controller 1854 may be configured to determine a state of the output voltage of the adjustment component 1860. When the first electric source system 1820 and the second electric source system 1830 include the anomaly, the controller 1854 may control an electrical conductance between the energy storage component 1852 and the load 1810 through the current adjustment component 1853.

The first electric source system 1820, the second electric source system 1830, and the third electric source system 1850 may be interlocked for power supply. That is, one of the first electric source system 1820, the second electric source system 1830, and the third electric source system 1850 may supply power to the load 1810.

In some embodiments, the current adjustment component 1853 may include an alter-direct conversion module 18531, a charging circuit 18532, a boost module 18533, and an inverter 18534. For example, the alter-direct conversion module 18531, the charging circuit 18532, the boost module 18533, and the inverter 18534 may be integrated into the current adjustment component 1853 (indicated as a dotted block in FIG. 19A). The alter-direct conversion module 18531 may be electrically connected with the controller 1854 and the switch 1851 (indicated as a dotted line in FIG. 19A). The charging circuit 18532 may be electrically connected with the alter-direct conversion module 18531, the energy storage component 1852, and the controller 1854 (indicated as a dotted line in FIG. 19A). The boost module 18533 may be electrically connected with the energy storage component 1852 and the controller 1854 (indicated as a dotted line in FIG. 19A). The inverter 18534 may be electrically connected with the alter-direct conversion module 18531, the boost module 18533, the controller 1854, and the load 1810.

The controller 1854 may be configured to determine and/or control an operation state of at least one of the alter-direct conversion module 18531, the charging circuit 18532, the boost module 18533, or the inverter 18534.

When the energy storage component 1852 needs to be powered, and one of the first electric source system 1820 and the second electric source system 1830 does not include the anomaly, the controller 1854 may control the switch 1851 to be electrically conducted with the alter-direct conversion module 18531. The controller 1854 may control the alter-direct conversion module 18531 to convert the inputted AC to the DC. The controller 1854 may control the inverter 18534 to convert the DC to the AC. The first electric source system 1820 or the second electric source system 1830 may provide power to the load 1810 through the control component 1840, the adjustment component 1860, the switch 1851, the alter-direct conversion module 18531, and the inverter 18534. At the same time, the controller 1854 may control the energy storage component 1852 to be in a charging mode, and the first electric source system 1820 or the second electric source system 1830 may provide power to the energy storage component 1852 through the control component 1840, the adjustment component 1860, the switch 1851, the alter-direct conversion module 18531, and the charging circuit 18532.

In some embodiments, each of the first electric source system 1820 and the second electric source system 1830 includes the anomaly, the controller 1854 may control the energy storage component 1852 to discharge and control the boost module 18533 to boost the output voltage of the energy storage component 1852. The energy storage component 1852 may provide power to the load 1810 through the boost module 18533 and the inverter 18534.

In some embodiments, each of the first electric source system 1820 and the second electric source system 1830 may not include the anomaly. One of the first electric source system 1820 and the second electric source system 1830 may provide power to the load 1810 through the control component 1840, the adjustment component 1860, and the switch 1851. For example, the controller 1854 may control the switch 1851 to be conducted with the load directly such that one of the first electric source system 1820 and the second electric source system 1830 may provide power to the load 1810 through the control component 1840, the adjustment component 1860, and the switch 1851. At the same time, another of the first electric source system 1820 and the second electric source system 1830 may provide power to the energy storage component 1852 through the current adjustment component 1853.

In some embodiments, the energy storage component 1852, the current adjustment component 1853, the switch 1851, and the controller 1854 may be integrated into a single electric source (i.e., the third electric source system 1850). In some embodiments, the energy storage component 1852, the current adjustment component 1853, the switch 1851, and the controller 1854 may be connected into the power supply system 1800, respectively.

In some embodiments, the first electric source system 1820 may further include mains electricity 1811, a surge protection circuit 1821, and a filter circuit 1822. The surge protection circuit 1821 may be connected with the mains electricity 1811. The filter circuit 1822 may be connected with the surge protection circuit 1821 and the control component 1840. The surge protection circuit 1821 and the filter circuit 1822 may be configured to smooth an output current of the mains electricity 1811.

In some embodiments, the surge protection circuit 1821 may include a discharge gap, a gas discharge tube, a thyristor, or the like, or any combination thereof. When there is no instantaneous overvoltage, the surge protection circuit 1821 may include a high impedance. When there is an instantaneous overvoltage (e.g., lightning), the impedance of the surge protection circuit 1821 may be reduced to a low value, thereby allowing a lightning current to pass.

The surge protection circuit 1821 may include a zinc oxide arrester, a varistor, a suppressor diode, an avalanche diode, or the like, or any combination thereof. When there is no instantaneous overvoltage, the surge protection circuit 1821 may include a high impedance. With an increase of surge current and voltage, the impedance of the surge protection circuit 1821 may continue to decrease. A current-voltage characteristic of the surge protection circuit 1821 may include a strongly nonlinear.

In some embodiments, before an interference circuit (e.g., the lightning current) damages one or more components of the movable medical device, the surge protection circuit 1821 and the filter circuit 1822 may lead the interference circuit to a ground line bypassing an undesirable power source (e.g., lightning) of the interference circuit to a neutral line.

Referring to FIG. 20, the second electric source system 1830 may further include a power generation component 1831. The power generation component 1831 may be connected with the control component 1840. If the first electric source system 1820 (e.g., the mains electricity 1811) includes the anomaly, the control component 1840 may be configured to control the power generation component 1831 to operate. In some embodiments, the control component 1840 may be configured to switch the power generation component 1831 to supply power to the load 1810 if the first electric source system 1820 (e.g., the mains electricity 1811) includes the anomaly.

The power generation component 1831 may include a diesel power generator, a photovoltaic power generator, a hydroelectric power generator, a hand-cranked power generator, or the like, or any combination thereof.

In some embodiments, the second electric source system 1830 may further include an energy storage component 1832 (also referred to as a second energy storage component 1832). The energy storage component 1832 may be electrically connected with the power generation component 1832 and the control component 1840. For example, the second energy storage component 1832 may be located in a space between the power generation component 1831 and the control component 1840. The second energy storage component 1832 may store the power generated by the power generation component 1831.

In some embodiments, the first electric source system 1820 may further include an energy storage component (also referred to as a third energy storage component). The third energy storage component may be connected with the filter circuit 1822 and the adjustment component 1860, respectively. The third energy storage component may be located in a space between the filter circuit 1822 and the adjustment component 1860.

In some embodiments, the power generation component 1831 may include a solar power generation component. The second energy storage component 1832 may include a combiner 18321, a controller 18322, an energy storage component 18323 (referred to as a second energy storage component or a charging circuit), and an inverter 18324. The combiner 18321 may be connected with the solar power generation component. The controller 18322 may be connected with the combiner 18321. The energy storage component 18323 may be connected with the controller 18322. The inverter 18324 may be connected with the controller 1840 and the energy storage component 18323.

The solar power generation component may include a plurality of battery panels. The plurality of battery panels may be configured to convert light energy into electric energy. The combiner 18321 may be configured to gather the scattered electric energy together. The controller 18322 may be configured to manage the charging and discharging of the energy storage component 18323 by the solar power generation component. The inverter 18324 may be configured to convert a direct current into a three-phase alternating current.

The power generation component 1831 may be disposed on a surface of the load 1810.

In some embodiments, the load 1810 may include a medical device in movable medical device. The second electric source system 1830 may include a power generator on the movable platform. The power generator on the movable platform may provide power for the movable platform and the load 1810.

It should be noted that the description of the power supply system 1800 is intended to be illustrative, and not to limit the scope of the present disclosure. Many alternatives, modifications, and variations will be apparent to those skilled in the art. The features, structures, methods, and other characteristics of the exemplary embodiments described herein may be combined in various ways to obtain additional and/or alternative exemplary embodiments. For example, the power supply system may include more than three electric source systems.

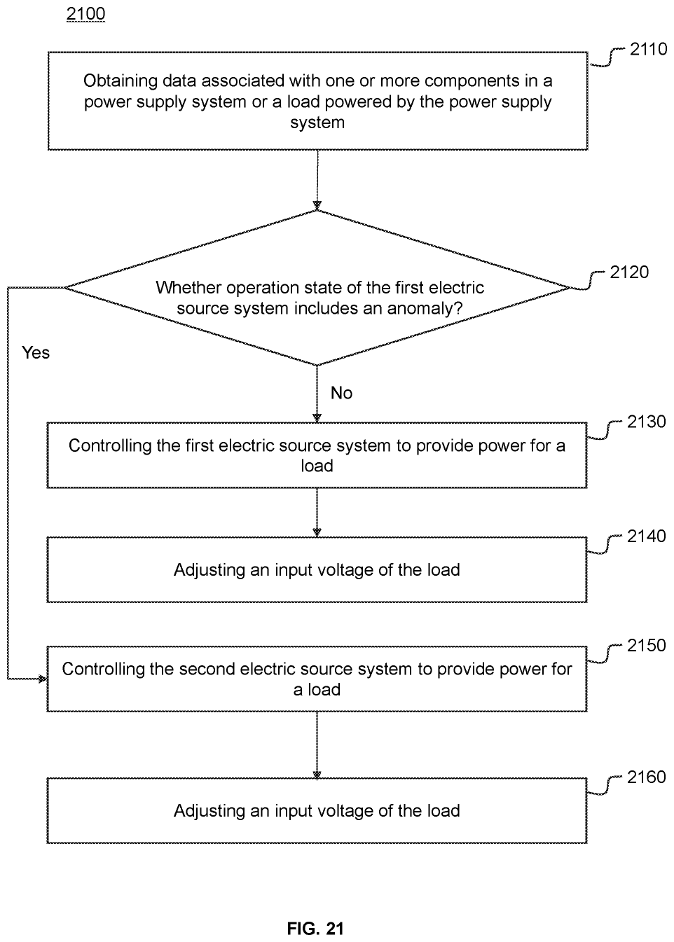

FIG. 21 is a flowchart illustrating an exemplary process for power supply according to some embodiments of the present disclosure. In some embodiments, process 2100 may be executed by the movable medical system (e.g., the movable medical system 100). For example, the process 2100 may be implemented as a set of instructions (e.g., an application) stored in a storage device (e.g., the storage device 130, the storage device 361, and/or the storage device 570). In some embodiments, the processing device 120, or the power supply system 1700 (e.g., electronic components 1740 illustrated in FIG. 17) may execute the set of instructions and may accordingly be directed to perform the process 2100. The operations of the illustrated process presented below are intended to be illustrative. In some embodiments, the process 2100 may be accomplished with one or more additional operations not described and/or without one or more of the operations discussed. Additionally, the order of the operations of process 2100 illustrated in FIG. 21 and described below is not intended to be limiting.

In 2110, the power supply system (e.g., the acquisition module 1742) may obtain data associated with one or more components in the power supply system or a load powered by the power supply system. The power supply system may include a first electric source system, a second electric source system, a control component, and an adjustment component. More descriptions for the power supply system may be found elsewhere in the present disclosure (e.g., FIGS. 17-20 and the descriptions thereof). The data associated with one or more components may include operation parameters (e.g., a voltage, a current, etc.) of one or more components (e.g., the first electric source system, the second electric source system). More descriptions regarding the obtaining of the data may be found elsewhere in the present disclosure (e.g., FIGS. 17-20 and the descriptions thereof).

In 2120, the power supply system (e.g., the control module 1744) may determine whether an operation state of the first electric source system includes an anomaly based on the acquired data. In response to determining that the first electric source system does not include an anomaly, the power supply system 1700 may perform operation 2130. In response to determining that the first electric source system includes an anomaly, the power supply system may perform operation 2150.

In 2130, the power supply system (e.g., the control module 1744) may control the first electric source system to provide power for a load, i.e., an electrical conductance between the first electric source system and the load. For example, the control module 1744 may control an electrical conductance between the first electric source system and the adjustment component such that the first electric source system may power the load through the adjustment component. As another example, the control module 1744 may control an electrical conductance between the first electric source system and load directly such that the first electric source system may power the load.

In 2140, the power supply system (e.g., the adjustment module 1746) may adjust an input voltage of the load. In some embodiments, the power supply system may adjust the input voltage of the load by adjusting an input voltage of the adjustment component (or the control component) and/or output voltage of the first electric source system (or the control component). In some embodiments, the adjustment module 1746 may acquire information associated with an output voltage (or current) of the first electric source system (or the control component) and/or the input voltage (or current) of the adjustment component (or the control component) when the first electric source system provides power for the load. The power supply system (e.g., the adjustment module 1746) may adjust output voltage (or current) of the first electric source system (or the control component) and/or the input voltage (or current) of the adjustment component (or the control component) to a preset voltage value or a preset voltage range based on the information associated with an output voltage (or current) of the first electric source system (or the control component) and/or the input voltage (or current) of the adjustment component (or the control component).

In 2150, the power supply system (e.g., the control module 1744) may control the second electric source system to provide power for the load, i.e., an electrical conductance between the second electric source system and the load. For example, the control module 1744 may control an electrical conductance between the second electric source system and the adjustment component such that the second electric source system may power the load through the adjustment component. As another example, the control module 1744 may control an electrical conductance between the second electric source system and load directly such that the first electric source system may power the load.

In 2160, the power supply system (e.g., the adjustment module 1746) may adjust an input voltage of the load. In some embodiments, the power supply system may adjust the input voltage of the load by adjusting the input voltage of the adjustment component (or the control component) and/or output voltage of the second electric source system (or the control component). In some embodiments, the adjustment module 1746 may acquire information associated with an output voltage (or current) of the second electric source system (or the control component) and/or the input voltage (or current) of the adjustment component (or the control component) when the second electric source system provides power for the load. The power supply system (e.g., the adjustment module 1746) may adjust output voltage (or current) of the second electric source system (or the control component) and/or the input voltage (or current) of the adjustment component (or the control component) to a preset voltage value or a preset voltage range based on the information associated with an output voltage (or current) of the second electric source system (or the control component) and/or the input voltage (or current) of the adjustment component (or the control component).

It should be noted that the above description is merely provided for the purposes of illustration, and not intended to limit the scope of the present disclosure. For persons having ordinary skills in the art, multiple variations and modifications may be made under the teachings of the present disclosure. However, those variations and modifications do not depart from the scope of the present disclosure. For example, the operation 2130 and the operation 2140 may be integrated into a single operation. As another example, the operation 2150 and the operation 2160 may be integrated into a single operation.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicate, propagate, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C#, VB. NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

1. A movable medical device, comprising:
a medical cabin configured to accommodate medical resources for providing multiple types of medical services; and
a movable platform configured to move the medical cabin to a desired location, wherein the medical cabin includes:
a radiation room configured to accommodate a radiation device for providing a medical imaging service;
a sample processing room configured to accommodate medical resources for providing at least one of a sampling service or a sample detection service; and
a control room configured to accommodate a control platform used to control the radiation device for providing the medical imaging service.

2. The movable medical device of claim 1, wherein the sample processing room includes:
a sampling window for an interaction between a service provider and a service receiver of the sampling service.

3. The movable medical device of claim 1, wherein the control room includes a first door, the radiation room includes a second door, and the sample processing room includes a third door.

4. The movable medical device of claim 3, wherein a distance between two of the first door, the second door, and the third door exceeds a threshold.

5. The movable medical device of claim 3, wherein an isolation device is provided in a space between two of the first door, the second door, and the third door, and configured to prevent personnel to pass through the space.

6. The movable medical device of claim 3, wherein the first door and the second door are located at different sides of the medical cabin.

7. The movable medical device of claim 1, wherein the radiation room includes one or more image acquisition devices configured to acquire image data of a service receiver in the radiation room, and the control platform is configured to control the radiation device for providing the medical imaging service based on the image data.

8. The movable medical device of claim 7, wherein the control platform is configured to:
determine, based on the image data, a scanning region of the service receiver; and
control, based on the scanning region, the medical imaging service to scan the service receiver.

9. The movable medical device of claim 8, wherein the control platform is configured to:
determine, based on the image data, at least one of a posture or a position of the service receiver on a couch;
determine whether the at least one of the posture or the position of the service receiver satisfies a condition; and
in response to determining that the at least one of the posture or the position of the service receiver does not satisfy the condition, provide a reminder for guiding the service receiver to adjust the at least one of the posture or the position.

10. The movable medical device of claim 1, wherein the radiation room includes a disinfection device to disinfect the radiation room according to one or more disinfection parameters.

11. The movable medical device of claim 10, wherein the one or more disinfection parameters are determined based on an input instruction of an operator in the control room or determined based on scanning data of the radiation device.

12-13. (canceled)

14. A movable medical device, comprising:
a medical cabin configured to accommodate medical resources for providing one or more types of medical services; and
a movable platform configured to move the medical cabin to a desired location, wherein the medical cabin includes:
a power supply system configured to provide power for a load in the movable medical device, wherein the power supply system includes:
a first electric source system and a second electric source system configured to provide power to the load;
a control component configured to determine an operation state of at least one of the first electric source system or the second electric source system, and control an operation of the at least one of the first electric source system or the second electric source system based on operation state.

15. The movable medical device of claim 14, wherein
the operation state of at least one of the first electric source system or the second electric source system indicates whether the first electric source system includes an anomaly, and
the control component is configured to:
in response to determining that the first electric source system does not include an anomaly, control the first electric source system to provide power for the load; or
in response to determining that the first electric source system includes an anomaly, control the second electric source system to provide power for the load.

16. The movable medical device of claim 14, wherein the power supply apparatus includes:
an adjustment component electrically connected with the load and the control component, and configured to adjust an input voltage of the load.

17-27. (canceled)

28. A movable medical device, comprising:
a medical cabin configured to accommodate medical resources for providing one or more types of medical services;
a movable platform configured to move the medical cabin to a desired location; and
a monitoring system including one or more monitoring devices and a control device, wherein
the one or more monitoring devices are configured to acquire a monitoring result related to at least one of an operation state of at least one of components of the movable medical device or an environment parameter where the medical cabin is located; and
the control device is configured to provide a feedback based on the monitoring result.

29. (canceled)

30. The movable medical device of claim 28, wherein the feedback includes at least one of prompt instruction associated with the monitoring result or an adjustment instruction for adjusting an operation of one or more components related to the monitoring result.

31-37. (canceled)

38. The movable medical device of claim 1, wherein the sample processing room includes a sampling window for an interaction between a service provider and a service receiver of the sampling service.

39. The movable medical device of claim 1, wherein the sample processing room includes at least one of:
a transmission channel through which an assistant transfers a sample to a disinfection region,
an inactivation device configured to inactivating the sample,
a biosafety cabinet configured to provide a safe environment for storing the sample, or
a sample analysis device for sample detection.

40. The movable medical device of claim 1, wherein the sample processing room includes a pressure adjustment device configured to adjust a gas pressure inside the sample processing room to be lower than a gas pressure outside the sample processing room.

41. The movable medical device of claim 3, wherein the third door and the second door are located at different sides of the medical cabin.
